(19) United States
(12) Patent Application Publication
(10) Pub. No.: US 2022/0177954 A1
(43) Pub. Date: Jun. 9, 2022

(54) NUCLEIC ACID PROBES

(71) Applicant: CYTOTEST INC., ROCKVILLE, MD (US)

(72) Inventor: Reinhard Ebner, ROCKVILLE, MD (US)

(21) Appl. No.: 17/542,078

(22) Filed: Dec. 3, 2021

Related U.S. Application Data
(60) Provisional application No. 63/121,115, filed on Dec. 3, 2020.
(60) Provisional application No. 63/133,123, filed on Dec. 31, 2020.

(51) Int. Cl. C12Q 1/6841; C12Q 1/6886; A61K 31/5025; A61K 31/502; A61K 38/14

(52) U.S. Cl. CPC C12Q 1/6841; C12Q 1/6886; A61K 31/5025; C12Q 2600/106; A61K 38/14; C12Q 2600/158; A61K 31/502

(57) ABSTRACT

A nucleic acid probe for hybridization to chromosomal DNA comprises at least two different nucleic acid molecules from the same chromosomal region of CHD1, where the nucleic acid molecules are present in different proportions from each other. A method of treating prostate cancer in a subject is also provided based on an assessment of whether the subject's prostate cancer is associated with CHD1 deficiency.

### BACKGROUND

African American (AA) men have significantly higher incidence and mortality rates from prostate cancer (PC) compared to individuals of European ancestry (EA)¹. Recent studies demonstrated that AA men are at higher risk of progression after radical prostatectomy, even in equal access settings and when accounting for socioeconomic status^{2,3}. While the reasons underlying these disparities are multifactorial, the data strongly argue that germline and/or somatic genetic differences between AA and EA men may in part explain these differences.

Comparative analysis of AA and EA prostate tumors have identified several genetic differences. PTEN deletions, ERG translocations and/or ERG over-expression are more frequent in PCs of EA men⁴⁻⁶. In contrast, LSAMP and ETV3 deletions, ZFHX3 mutations, MYC and CCND1 amplifications and KMT2D truncations are more frequent in PCs of AA men⁷⁻⁹. ERF, an ETS transcriptional repressor, also showed an increased mutational frequency in AA prostate cancer cases with probable functional consequences such as increased anchorage independent growthm, and SPINK1 expression is also enriched in African American PC¹¹.

Chromodomain helicase DNA-binding protein 1 (CHD1) deletion is frequently present in prostate cancer. Deletions are associated with increased Gleason score and faster biochemical recurrence¹², activation of transcriptional programs that drive prostate tumorigenesis¹³ and enzalutamide resistance¹⁴. Mechanistically, CHD1 loss influences prostate cancer biology in at least two ways. CHD1, an ATPase-dependent chromatin remodeler, contributes to a specific distribution of androgen receptor (AR) binding in the genome of prostate tissue. When lost, the AR cistrome redistributes to HOXB13 enriched sites and thus alters the transcriptional program of prostate cancer cells¹³. CHD1 also contributes to genome integrity. It is required for the recruitment of CtIP, an exonuclease, to DNA double strand breaks (DSB) to initiate end resection. Upon CHD1 loss this important step in DSB repair is impaired, leading to homologous recombination deficiency^{15,16}. The functional impact of CHD1 loss is further influenced by the presence of SPOP mutations, which were reported to be associated with the suppression of DNA repair¹⁷.

CHD1 loss is frequently subclonal¹⁸, which makes its detection by next generation sequencing more challenging¹⁹, and it may go undetected depending on the subclonal fraction of cells harboring this aberration. Therefore, the true proportion of PC cases with CHD1 may be underestimated. Thus, the frequency of CHD1 loss in EA and AA PC was investigated by methods more sensitive to detecting subclonal deletions including evaluations of multiple tumor foci present in each prostatectomy specimen.

### SUMMARY

In one aspect, the present disclosure provides a nucleic acid probe for hybridization to chromosomal DNA, comprising at least two different nucleic acid molecules from the same chromosomal region of CHD1, where said nucleic acid molecules are present in different proportions from each other. In some embodiments, the probe comprises at least 10 nucleic acid molecules, where at least three of said molecules are present in different proportions from each other and in different proportions that they occur in nature.

In some embodiments, the probe is for detecting a deletion in the CHD1 gene and the probe comprises molecules having sequences from the CHD1 gene, where the molecules are present in different proportions from each other and in different proportions than those in which they occur in nature.

In some embodiments, the probe is for detecting a deletion in the CHD1 gene and the probe comprises molecules having sequences from the CHD1 gene, where the molecules are present in different proportions from each other and in different proportions that they occur in nature.

In some embodiments, the probe comprises molecules having sequences from the LSAMP gene deleted in patients with prostate cancer, where the molecules are present in different proportions from each other and in different proportions that they occur in nature.

In some embodiments, the probe further comprises molecules having sequences from the PTEN gene deleted in patients with prostate cancer, where the molecules are present in different proportions from each other and in different proportions that they occur in nature.

In some embodiments, the probe further comprises at least two molecules having sequences identified in FIG. 3.

In some embodiments, the probe further comprises at least two molecules having sequences from Table 1.

In some embodiments, the probe further comprises five molecules having sequences from Table 1.

In another aspect, a method of treating prostate cancer in a subject is provided, comprising: assessing whether the subject's prostate cancer is associated with CHD1 deficiency (such as CHD1 deletion) by performing a FISH assay using a nucleic acid probe disclosed herein on a nucleic acids sample obtained from the subject; and if it is determined that the subject's prostate cancer is associated with CHD1 deficiency, administering an effective amount of at least one of talazoparib and Olaparib to the subject.

In another aspect, a method of treating prostate cancer in a subject is provided, comprising: assessing whether the subject's prostate cancer is associated with CHD1 deficiency based on a nucleic acids sample obtained from the subject; and if it is determined that the subject's prostate cancer is associated with CHD1 deficiency, administering an effective amount of bleomycin to the subject. In some embodiments, the method further comprises administering to the subject a PARP inhibitor, such as Olaparib and Talazoparib.

In these methods, the nucleic acids sample can be obtained from a cancerous prostate tissue of the subject, or obtained from circulating tumor cells or cell-free circulating nucleic acids from the subject.

### BRIEF DESCRIPTION OF THE DRAWINGS

Examples of several of the various embodiments of the present invention are described herein with reference to the drawings.

FIG. 1A shows a target region of CHD1 on chromosome 5.

FIG. 1B shows the positions of D5S23 and D5S721 (F is forward primer and R is reverse primer), position markers which overlap with the SEMA5A gene on chromosome 5.

FIG. 2 shows a commonly deleted region of the CHD1 from 93.5-104 Mbpa.

FIG. 3 is a list of candidate clones useful in the design of probes to detect CHD1 deletions.

FIG. 4 is a list of genomic clones for the design of control probes matching the D5S23 and D5S721 genomic region.

FIG. 5a shows FISH assay results of prostate cancer cells harboring mono-allelic deletion for CHD1 (upper left) vs. prostate cancer cells with wild type (diploid) CHD1 (upper right) are visualized by FISH assay. Representative view fields capture 3-3 cell nuclei at 60× magnification. Inset table summarizes the higher frequency of CHD1 deletion in prostatic carcinoma of AA vs. EA patients.

FIG. 5b shows FISH assay results of multiple tumor samples from certain patients. BCR: univariable Kaplan-Meier curve; Metastasis: univariable Kaplan-Meier curve. Multiple tumor samples from 200 patients were assessed by FISH assay that identified 41 patients with CHD1 deletion (left panel). The heatmap depicts the sampled largest tumor 1 (T1), second largest tumor (T2), and so on. Numbers denote pathological Gleason grade for each tumor.

FIG. 5c shows univariable Kaplan-Meier curves of BCR and Metastasis.

FIG. 6 shows HRD markers in the PRAD WGS. a) HRD-score, the sum of the three genomic scars, HRD-LOH, LST, and ntAI. b) number of somatic mutations contributable to single-base substitution signature 3, c) number of structural variants contributable to rearrangement signature 5.

FIG. 7 shows somatic signatures in cell-line data. a) Single Nucleotide Substitution (SBS) signatures, b) Indel signatures, c) Rearrangement signatures. The number of mutations indicated originate from the reconstructed mutational spectra.

FIG. 8a shows min-max normalized HRD-related attributes of the PRAD WGS samples.

FIG. 8b shows proportion of tumor cells with intact CHD1.

FIG. 8c shows sensitivity of CHD1 wild type and CHD1 deficient cells to PARP inhibitors Olaparib and Talazoparib.

FIG. 8d shows Bleomycin sensitivity in PC-2 cell lines.

### DETAILED DESCRIPTION

Embodiments relate to nucleic acid probes for detecting nucleic acid sequences. The sequences can be present in any sample, including in tissues, cells, organelles, chromosomes, biopsy samples, tissues present on slides, skin, hair, environmental, soil, clothing, forensic samples, etc. Present embodiments particularly relate to the detection of deletions in the CHD1 gene, alone, or in combination with other genes, such as deletions in the LSAMP and PTEN genes.

Generally, a target acid nucleic acid may be selected for detection. For example, it may be desired to determine whether a gene has been amplified, rearranged or deleted in a chromosome, or translocated to another chromosome. Genes may also become rearranged, including by deletions, insertions, fusions, translocations, and other aberrations, e.g., involving other genes and chromosomal regions.

The probes of the present embodiments are useful for detecting any of the above-mentioned genomic changes in a genome. In addition, the probes are useful in detecting aneuploidy, such as trisomy, where karyotyping is typically utilized to detect genetic abnormalities.

There are a variety of different sources from which the nucleic acid can be obtained. These include, but are not limited to: BAC (bacterial artificial chromosome) libraries, YAC (yeast artificial chromosome) libraries, PCR (polymerase chain reaction) product fragments, bacteriophage libraries, plasmid libraries, cDNA libraries, genomic libraries, libraries made from dissected chromosomal regions.

The probe may be prepared by any suitable method. Generally, once the nucleic acid to be used as a probe source is obtained, it will be amplified to increase its amount, e.g., by PCR, nick-translation, random priming, etc.

Probes prepared in accordance with the above-mentioned methods may incorporate naturally-occurring and non-naturally occurring nucleotides. Examples of non-naturally occurring nucleotides useful in the present include, but are not limited to: nucleotides which are disclosed in, for example, U.S. Pat. Nos. 5,476,928, 5,449,767, and 5,328,824.

The probes may be labeled with detectable labels to enable detection of the probe. The probe may be labeled prior to its hybridization with a target, during hybridization, or after hybridization. Detectable labels and methods of labeling nucleic probes are well known in the art.

Useful detectable labels include, but are not limited to: fluorescent dyes, biotin, enzymes, fluorescein, Texas Red, DNP, fucose. Labeling methods are well-known in the art.

The nucleic acid probes of the present embodiments are non-naturally occurring. Specifically, in example embodiments, the probes are not directed to contiguous and connected chromosomal regions, but rather are fragmented portions of the desired region. For example, for region 5q15-q21.1 deletions, the probe may not comprise molecules which are continuous or contiguous with a genomic sequence from that region, but rather contains non-continuous fragments from it.

In addition to not being a continuous region, the probe may not contain equal representations or proportions of each sub-region within the target region. For example, if chromosome band 5q15-q21.1 comprising the CHD1 gene is selected, the probe may contain fragments of it in unequal quantities, i.e., if the region has ten different fragments within it, fragment 1 may be present in 1× quantity, fragment 2 in 2× quantity, fragment 3 in 3× quantity, fragment 4 in 4× quantity, and so on. Such unequal representations from the molecules as they occur in nature may result from the selection of non-overlapping molecules from which to prepare the probe, and subsequent amplification reactions which unequally amplify parts of the target nucleic acid.

### Hybridization

Once a probe is produced as described above, it may be used to detect the target nucleic acid in a sample. Generally, the probe may be used as a hybridization probe in any suitable format. Formats include, without limitation, liquid hybridization, PCR, Southern, Northern, microarrays, microscope slides, paraffin sections, and cryosections.

Hybridization conditions are well known in the art. See, e.g., Wangsa et al., Am. J. Pathol., 175(6): 2637-2645, December 2009.

As indicated above, the probe may be pre-labeled, such that after hybridization is complete and unbound probe is washed away, the probe can be immediately detected. In another embodiment, detectable label may be added to the probe after its bound to the target nucleic acid.

### In Situ Hybridization

In situ hybridization (ISH) is a technique that involves hybridizing a probe to a target nucleic acid in which the target is present in a tissue section (paraffin, plastic, cryo, etc.), cells, embryos, etc. In this technique, the target is detected in situ in the location where it is normally found. For example, the target may be detected in the cell cytoplasm, in an organelle (e.g., mitochondria), or in the chromosomal DNA. The chromosomal DNA in general is an intact chromosome that can be present in the tissue section or cell in its intact form or it can be isolated. In each case, the sample containing the target is treated in such a way that the probe can access the target chromosome or chromosome fragment, hybridize to it, and then be detected. When the probe is fluorescently labeled, the technique is known as fluorescence in situ hybridization (FISH).

FISH can be performed with one or more detectable labels. For example, M-FISH (multi-fluor or multi-color or multispectral FISH) is a technique in which multiple probes, each of which binds to a different DNA sequence and each of which bears a different detectable label, is used to detect multiple different sequences on the same sample, for example, on the same chromosome. M-FISH is useful for looking at chromosome rearrangements or translocations, or looking at independent loci in the same sample. See, e.g., U.S. Pat. No. 5,880,473 for the use of multiple filters in M-FISH. For SKY (spectral karyotyping), in which each chromosome pair is visualized in a different color, see, e.g., Schröck E, du Manoir S, Veldman T, Schoell B, Wienberg J, Ferguson-Smith Mass., Ning Y, Ledbetter D H, Bar-Am I, Soenksen D, Garini Y, Ried T. Multicolor spectral karyotyping of human chromosomes. Science 273:494-497, 1996.

A given dye is characterized by an excitation (absorption) spectrum and an emission spectrum. The excitation and emission spectra are also sometimes referred to as the excitation and emission bands. When the dye is irradiated with light at a wavelength within the excitation band, the dye fluoresces, emitting light at wavelengths in the emission band.

Thus, when the sample is irradiated with excitation radiation in a frequency band that excites a given dye, portions of the sample to which the probe labeled with the given dye is attached fluoresce. If the light emanating from the sample is filtered to reject light outside the given dye's emission band, and then imaged, the image nominally shows only those portions of the sample that bind the probe labeled with the given dye.

The term “hybridization” refers to the specific binding of a nucleic acid to a complementary nucleic acid via Watson-Crick base pairing. The term “in situ hybridization” refers to specific binding of a nucleic acid to a target nucleic acid in its normal place in a sample, such as on metaphase or interphase chromosomes. The terms “hybridizing” and “binding” are used interchangeably to mean specific binding between a nucleic acid probe and its complementary sequence.

The term “chromosomal region” means a contiguous length of nucleotides in the genome of an organism. A chromosomal region may be in the range of 10 kb in length to less than a complete chromosome of an entire chromosome, e.g., 100 kb to 10 MB for example.

FISH probes are most typically in the 50 kpb to 1000 kbp length range.

The term “in situ hybridization conditions” refers to conditions that facilitate hybridization of a nucleic acid to a complementary nucleic acid in an intact chromosome. Suitable in situ hybridization conditions may include both hybridization conditions and optional wash conditions, which include temperature, concentration of denaturing reagents, salts, incubation time, etc. Such conditions are known in the art.

FISH probes can be prepared according to standard procedures. See, e.g., Bolland, D. J., King, M. R., Reik, W., Corcoran, A. E., Krueger, C. Robust 3D DNA FISH Using Directly Labeled Probes. J. Vis. Exp. (78), e50587, doi:10.3791/50587 (2013).

### Probe Selection

Determination of the specific probe to be used to detect the target sequence can be accomplished routinely. Probe property may be selected based on one or more of the following factors: duplex melting temperature, hairpin stability, GC content, probe complementary to an exon, probe complementary to a gene, probe complementary to intron, probe complementary to multiple regions in the genome, and a proximity score. In certain embodiments, the probes can be comprised of fragments which were selected for different properties, such as the factors mentioned above. For example, fragments can be selected based on different factors, such as GC content or hairpin stability, and then pooled to make the final nucleic acid probe. See US 2011/003935 A1 for methods of selecting probes.

When a certain chromosomal region is targeted, a set of tiled or overlapping candidate nucleic acids can be selected, such as tiled YAC or BAC clones. Such tiled or overlapping nucleic acids can be constructed to unique sequences in the desired chromosomal regions. Because of the tiling or overlapping, the regions of overlap are in greater quantity than other non-overlapping regions, and thus are represented in higher amounts than in the native chromosome, particularly when amplified using a polymerase or other amplification method.

When ISH probes are made from artificial chromosomes, such as yeast artificial chromosomes (YAC), bacterial artificial chromosomes (BAC) and phage artificial chromosomes (PAC), etc., nucleotide repeats and repetitive sequences are usually present which can produce non-specific fluorescent signal and reduce the ISH detection specificity and sensitivity. Methods to reduce hybridization are known in the art, and include adding repetitive sequences to the hybridization mixture or making ISH probes that lack such sequences.

The probes can be tested to avoid using probes hybridizing to repetitive and repeat sequences. Probes can be produced using sets of various oligonucleotides which avoid repetitive sequences present in a flanking region. Such sets can be distinctly labeled, with separate or distinct reporter molecules for each probe (or set of oligonucleotides) that is aimed at the respective flanking region. Such probes can each consist of multiple labeled oligonucleotides, each hybridizing to a distinct area in a region which lacks repetitive sequences. One probe can, for example, contain from 10 up to 200 of such oligonucleotides, preferably from 50-150, each oligonucleotide, for example, being 10-20 nucleotides long.

As mentioned, the probes of present embodiments may be produced by any suitable or known method. For example, probes may be produced using set of oligonucleotides that amplify unique, non-repetitive regions. See, e.g., WO 2014036525 A1.

Probes designed for translocations, break points, inversions, and other chromosomal rearrangements can be produced routinely. Generally, chromosomal regions flanking a breakpoint are selected. Each flanking region is labeled differently.

Probes can also be provided to identify translocations. In such cases, a balanced pair of nucleic acid probes can be produced. The probes in said pair are comparable or balanced in that they are designed to be of, for example, comparable size or genomic length with the final aim of facilitating the generation of signals of comparable intensity. In addition, said probes can be comparably labelled with reporter molecules resulting in signals of comparable intensity. In addition, said probes can each be labelled with a different fluorochrome, facilitating detection on one spot of different color when they co-localize when no aberration is detected. In addition, probes can be selected to react with a chromosome, at respective complementary sites that are located at comparable distances at each side of a breakpoint or breakpoint cluster region of a chromosome. The distinct and balanced pair of nucleic acid probes provided by embodiments entails probes that are for example of comparable size or genomic length, each probe of the pair for example being from 1 to 10 kb, or 7 to 15 kb, or 10 to 20 kb, or 15 to 30 kb, or 20 to 40 kb, or 30 to 50 kb, or 40 to 60 kb, or 50 to 70 kb, or 60 to 80 kb, or 70 to 90 kb, or 80 to 100 kb, or 100 to 500 kb or more in length. By using such a distinct and balanced pair of probes flanking a breakpoint region and not overlapping the corresponding fusion region, false-positive diagnosis in hybridization studies is avoided.

### Labeling

The labeling may be done in any convenient way. For example, in certain cases, the probes may be labeled by chemically conjugating one or more labels to the one or more double stranded polynucleotides, e.g., using the Universal Linkage System (ULS™, KREATECH Diagnostics; van Gijlswijk et al., Universal Linkage System: versatile nucleic acid labeling technique Expert Rev. Mol. Diagn. 2001 1:81-91). Alternatively, the labeling may be done using nick translation, by random priming, or any other suitable method described in Ausubel et al. (Short Protocols in Molecular Biology, 3rd ed., Wiley & Sons, 1995) or Sambrook et al. (Molecular Cloning: A Laboratory Manual, Third Edition, (2001) Cold Spring Harbor, N.Y.). In certain cases, the one or more double stranded polynucleotides are labeled at multiple sites and not labeled by end labeling. As would be apparent embodiments of the method that use other labeling methods (e.g., nick translation or random priming) will produce products that differ in sequence and representation of sequence.

### CHD1 Probes

CHD1 is a member of a family of proteins that is characterized by the presence of chromo (chromatin organization modifier) domains and SNF2-related helicase/ATPase domains. CHD1 encodes a chromatin-remodeling enzyme composed of 1710 amino acids. See www.ncbi.nlm.nih.gov/gene/1105. The CHD genes are believed to alter gene expression by modifying the chromatin structure and thereby altering access of the transcriptional apparatus to its chromosomal DNA template. Id.

CHD1 gene is located on chromosome 5 at 5q15-q21.1. The gene maps to 98,189,689-98,264,661 in GRCh37 coordinates. The gene encodes protein products that correspond to the following UniProtKB identifiers: 014646. The complete RNA sequence is identified at GenBank: AF006513.1. Various variants of CHD1 are known.

Deletions in CHD1 gene are associated with cancer and other diseases. The probes of the present embodiment are able to detect such deletions, and are useful in predicting the occurrence of the disease, disease progression, and therapeutic interventions. For example, the presence of a CHD1 deletion indicates that the cancer is aggressive and requires early treatment.

CHD1 deletions associated with cancer and other diseases often co-occur with deletions and mutations in other genes and can be used to predict disease progression and drug sensitivity. The present embodiment relates to probes which are useful for detecting deletions and mutations in CHD1 gene in combination with other genes, e.g., to produce CHD1 genomic signatures.

To produce probes against CHD1 (where the absence of binding indicates the presence of a deletion), overlapping and non-overlapping chromosomal segments may be selected and routinely amplified to produce a non-naturally occurring probe composition. As indicted above, nucleic acid to produce such probes can be obtained from any suitable source, such as a BAC clones, YAC clones libraries, etc. One or more, such as e.g. 2, 3, 5, 7, 10 clones can be pooled together to create a probe. The clones can be amplified separately or pooled and then amplified. When non-overlapping clones are utilized, the clones can be selected such that the entire gene is represented, or only a part of it, where the clones collectively lack parts of the gene due to the selection of non-overlapping segments.

To optimize the signal intensity to facilitate FISH visualization while preserving high specificity, it was unexpectedly discovered that sets of five partially overlapping genomic clones, and greater coverage of the 3′ than the 5′ genomic regions as disclosed in Table 1, led to most desirable probe performance and most easy and concise results interpretation.

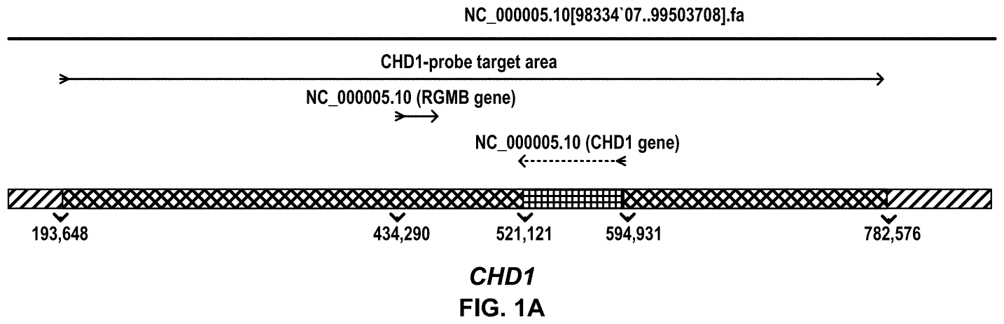

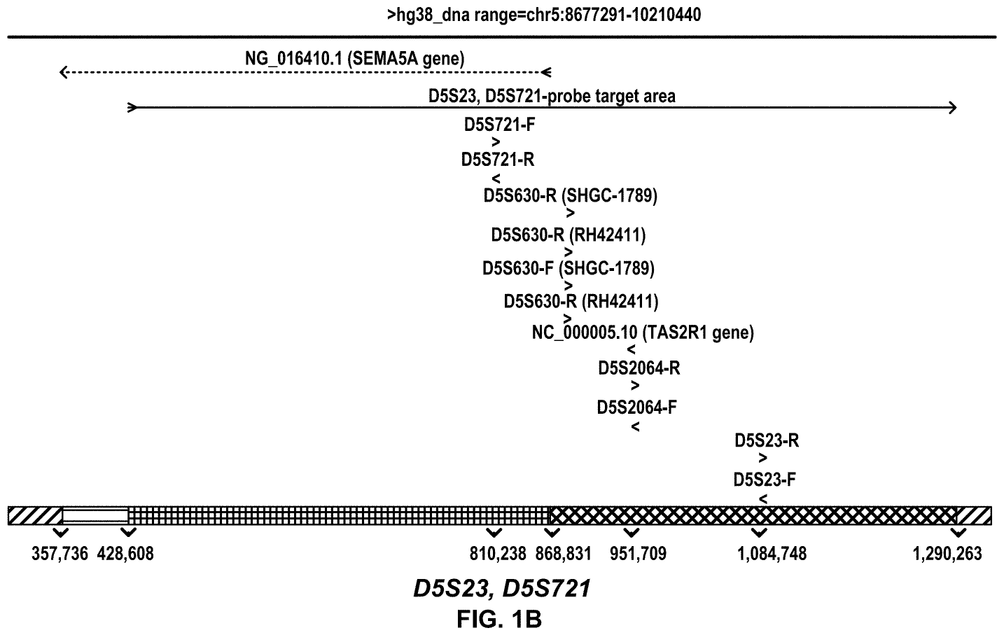

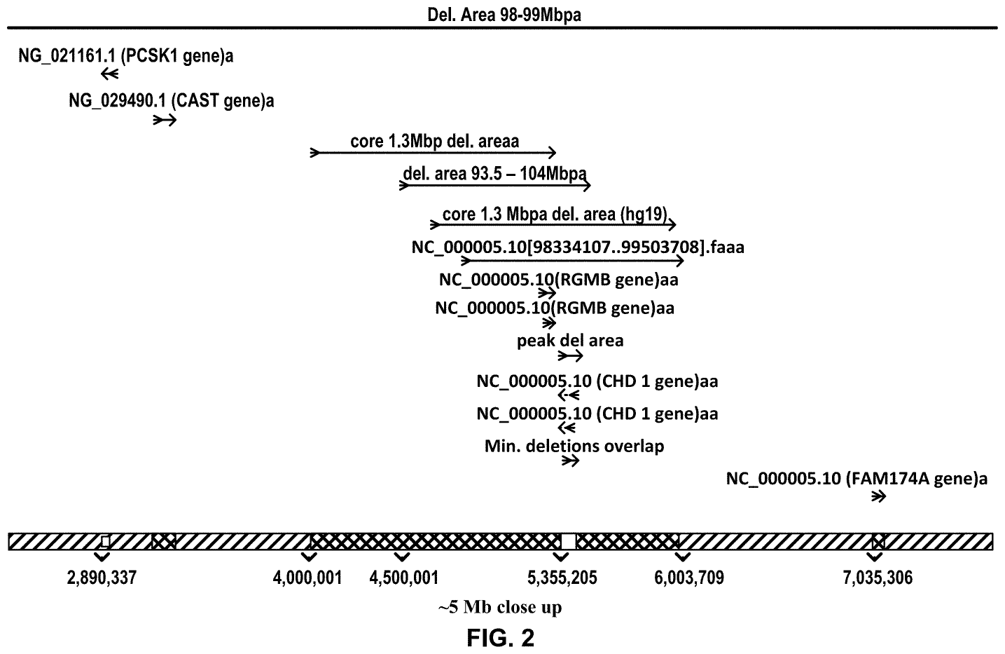

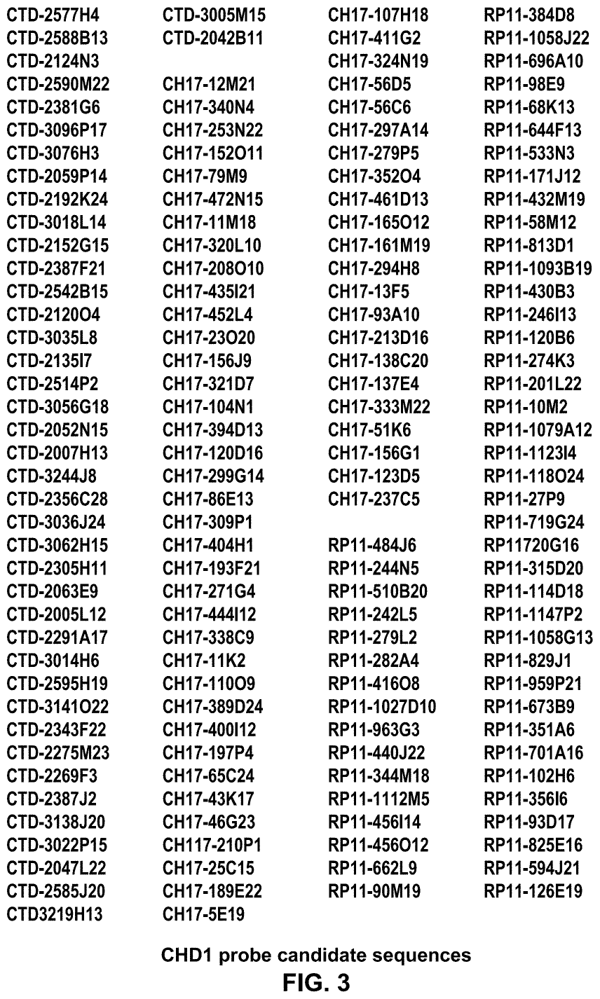

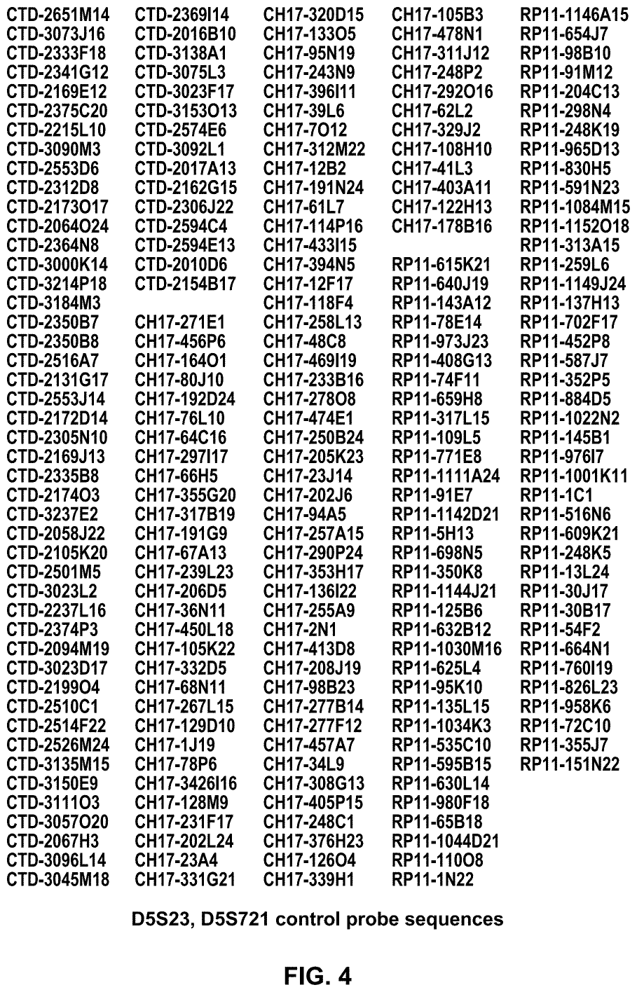

FIG. 1 is a CHD1 probe overview map. FIG. 1Aa shows the target region of CHD1 on chromosome 5. NC_000005.10 is the region of chromosome 5 which contains the CHD1 gene. FIG. 1B shows the positions of D5S23 and D5S721 (F is forward primer and R is reverse primer), position markers which overlap with the SEMA5A gene on chromosome 5. The D5630 and D5S2064 are other position markers for reference. The control probes (FIG. 4) contain both of the position markers D5S23 and D5S721 and are useful for normalizing gene copy number of chromosome 5. FIG. 2 shows a commonly deleted region of the CHD1 from 93.5-104 Mbpa. The highlighted portions show reported deletions within the larger area of 93.5-104 Mbpa. The “NC” identifiers refer to GenBank identifiers. The “peak del area” is the peak of where the reported deletions are identified. FIG. 3 is a list of candidate clones useful in the design of probes to detect CHD1 deletions. The numbers refer to genomic clones which are bacterial artificial chromosomes (BAC). The clones are listed on the NCBI “CloneDB” at https://www.ncbi.nlm.nih.gov/clone. The database lists the distributors where the clones can be obtained. The clones can be used singly, two or more, three or more, preferably four, and more preferably used as a combination of five or six probes to detect a CHD1 deletion.

FIG. 3 is showing a list of publicly available BAC clones that include sequences from CHD1 which are commonly deleted. The sequences and clones are incorporated by reference. Two or more clones can be selected to make a probe, e.g., where the clones are amplified separately or in combination by nick-translation, random primer, etc. All combinations of clones to make CHD1 probes are covered by the present embodiments. Table 1 lists preferred combinations.

The probes can be useful to detect amplification of the CHD1 gene or deletion of the gene. For example, gene deletion occurs in certain prostate cancers and therefore proves suitable to detect gene deletions, and as such are useful for diagnostic purposes. Accordingly, the embodiment comprises a method for detecting the presence or absence of CHD1 in a biological sample comprising nucleic acid. ISH probes are particularly useful for this purpose.

Detecting of abnormalities in the genomic regions of the CHD1 gene, particularly deletions within the gene, is also important in PTEN-deleted cancers, such prostate and breast cancers. It has been shown that CHD1 depletion specifically suppressed cell proliferation, survival and tumorigenic potential in PTEN deficient cancers (Zhao et al.). Deletion detection of CHD1 in both PTEN deleted and non-deleted patient groups is therefore beneficial, since in PTEN deleted cases CHD1 is essential, and thus its deletion indicates poor prognosis; whereas in PTEN undeleted cases CHD1-directed therapy may prove less effective.

PTEN deletions can be detected with probes. PTEN deletions are described in U.S. patent application Ser. No. 15/862,343 (published as 2018/0135137 A1), which is incorporated by reference.

It has also been reported by that deletions in CHD1 and MAP3K7 occur in cancer, particularly prostate cancers, and indicate more aggressive disease (Rodrigues et al., Cancer Res. 2015 Mar. 15; 75(6): 1021-1034). Thus, detection of both deletions is an important prognostic marker for disease status and prognosis.

Mutations and gene arrangement of the ERG gene have also been described associated with CHD1 gene deletions, such as TMPRSS2-ERG translocations (Tereshchenko et al. (Prostate. 2014 November; 74(15): 1551-1559), making both markers significant in disease assessment.

Chromosomal translocations of CHD1 have also been shown to result in the cancer. For example, Yao et al. (Molecular Cancer (2015) 14:81) reported translocations between the RUNX1 gene and CHD1 associated with hematopoietic cancer, particularly myeloid leukemia with t(5;21)(q21;q22).

With regard to prostate cancer, detection of CHD1 deletion can be combined with mutational/gene deletion detection of any of the known genes associated with prostate cancer and prostate cancer progression, such as, but not limited to LSAMP (e.g. Mikhaylenko et al.), mtDNA, PCA3, TMPRSS2-ERG, SPOP, FOXA1, PTEN, AR, HSD3B1, ZBTB16, IDH1, TP53, R131, AURKA, MYCN, NCOA1, PTK2, and YWHAZ. See Mikhaylenko et al. (Current Genomics, 2017, 18, 236-243); Bova et al. (2016 Cold Spring Harb Mol Case Stud 2: a000752). For example, metastatic tumors have been shown to exhibit homozygous deletion of TP53, hemizygous deletion of RB1 and CHD1, and amplification of FGFR1 (Bova et al.)

Present embodiments relate to probes for the CHD1 gene, particularly probes which are able to detect deletions in the gene, such as FISH probes. Useful probes comprise 1 to 6, or more of the BAC sequences listed in Table 1 below.

TABLE 1
CHD1 FISH Probe candidate clone combinations

| | | | | |
|---|---|---|---|---|
| clone set: | RP11-720G19 | RP11-430B3 | RP11-644F13 | |
| clone set: | RP11-720G19 | RP11-430B3 | RP11-264K18 | |
| clone set: | RP11-720G19 | RP11-430B3 | RP11-416O8 | |
| clone set: | RP11-720G19 | RP11-430B3 | RP11-90M19 | |
| clone set: | RP11-720G19 | RP11-430B3 | RP11-662L9 | |
| clone set: | RP11-720G19 | RP11-98E9 | RP11-644F13 | |
| clone set: | RP11-720G19 | RP11-98E9 | RP11-264K18 | |
| clone set: | RP11-720G19 | RP11-98E9 | RP11-416O8 | |
| clone set: | RP11-720G19 | RP11-98E9 | RP11-90M19 | |
| clone set: | RP11-720G19 | RP11-98E9 | RP11-662L9 | |
| clone set: | RP11-720G19 | RP11-58M12 | RP11-644F13 | |
| clone set: | RP11-720G19 | RP11-58M12 | RP11-264K18 | |
| clone set: | RP11-720G19 | RP11-58M12 | RP11-416O8 | |
| clone set: | RP11-720G19 | RP11-58M12 | RP11-90M19 | |
| clone set: | RP11-720G19 | RP11-58M12 | RP11-662L9 | |
| clone set: | RP11-720G19 | RP11-384D8 | RP11-644F13 | |
| clone set: | RP11-720G19 | RP11-384D8 | RP11-264K18 | |
| clone set: | RP11-720G19 | RP11-384D8 | RP11-416O8 | |
| clone set: | RP11-720G19 | RP11-384D8 | RP11-90M19 | |
| clone set: | RP11-720G19 | RP11-384D8 | RP11-662L9 | |
| clone set: | RP11-720G19 | RP11-533M23 | RP11-644F13 | |
| clone set: | RP11-720G19 | RP11-533M23 | RP11-264K18 | |
| clone set: | RP11-720G19 | RP11-533M23 | RP11-416O8 | |
| clone set: | RP11-720G19 | RP11-533M23 | RP11-90M19 | |
| clone set: | RP11-720G19 | RP11-533M23 | RP11-662L9 | |
| clone set: | RP11-719G24 | RP11-430B3 | RP11-644F13 | |
| clone set: | RP11-719G24 | RP11-430B3 | RP11-264K18 | |
| clone set: | RP11-719G24 | RP11-430B3 | RP11-416O8 | |
| clone set: | RP11-719G24 | RP11-430B3 | RP11-90M19 | |
| clone set: | RP11-719G24 | RP11-430B3 | RP11-662L9 | |
| clone set: | RP11-719G24 | RP11-98E9 | RP11-644F13 | |
| clone set: | RP11-719G24 | RP11-98E9 | RP11-264K18 | |
| clone set: | RP11-719G24 | RP11-98E9 | RP11-416O8 | |
| clone set: | RP11-719G24 | RP11-98E9 | RP11-90M19 | |
| clone set: | RP11-719G24 | RP11-98E9 | RP11-662L9 | |
| clone set: | RP11-719G24 | RP11-58M12 | RP11-644F13 | |
| clone set: | RP11-719G24 | RP11-58M12 | RP11-264K18 | |
| clone set: | RP11-719G24 | RP11-58M12 | RP11-416O8 | |
| clone set: | RP11-719G24 | RP11-58M12 | RP11-90M19 | |
| clone set: | RP11-719G24 | RP11-58M12 | RP11-662L9 | |
| clone set: | RP11-719G24 | RP11-384D8 | RP11-644F13 | |
| clone set: | RP11-719G24 | RP11-384D8 | RP11-264K18 | |
| clone set: | RP11-719G24 | RP11-384D8 | RP11-416O8 | |
| clone set: | RP11-719G24 | RP11-384D8 | RP11-90M19 | |
| clone set: | RP11-719G24 | RP11-384D8 | RP11-662L9 | |
| clone set: | RP11-719G24 | RP11-533M23 | RP11-644F13 | |
| clone set: | RP11-719G24 | RP11-533M23 | RP11-264K18 | |
| clone set: | RP11-719G24 | RP11-533M23 | RP11-416O8 | |
| clone set: | RP11-719G24 | RP11-533M23 | RP11-90M19 | |
| clone set: | RP11-719G24 | RP11-533M23 | RP11-662L9 | |
| clone set: | RP11-1093B19 | RP11-430B3 | RP11-644F13 | |
| clone set: | RP11-1093B19 | RP11-430B3 | RP11-264K18 | |
| clone set: | RP11-1093B19 | RP11-430B3 | RP11-416O8 | |
| clone set: | RP11-1093B19 | RP11-430B3 | RP11-90M19 | |
| clone set: | RP11-1093B19 | RP11-430B3 | RP11-662L9 | |
| clone set: | RP11-1093B19 | RP11-98E9 | RP11-644F13 | |
| clone set: | RP11-1093B19 | RP11-98E9 | RP11-264K18 | |
| clone set: | RP11-1093B19 | RP11-98E9 | RP11-416O8 | |
| clone set: | RP11-1093B19 | RP11-98E9 | RP11-90M19 | |
| clone set: | RP11-1093B19 | RP11-98E9 | RP11-662L9 | |
| clone set: | RP11-1093B19 | RP11-58M12 | RP11-644F13 | |
| clone set: | RP11-1093B19 | RP11-58M12 | RP11-264K18 | |
| clone set: | RP11-1093B19 | RP11-58M12 | RP11-416O8 | |
| clone set: | RP11-1093B19 | RP11-58M12 | RP11-90M19 | |
| clone set: | RP11-1093B19 | RP11-58M12 | RP11-662L9 | |
| clone set: | RP11-1093B19 | RP11-384D8 | RP11-644F13 | |
| clone set: | RP11-1093B19 | RP11-384D8 | RP11-264K18 | |
| clone set: | RP11-1093B19 | RP11-384D8 | RP11-416O8 | |
| clone set: | RP11-1093B19 | RP11-384D8 | RP11-90M19 | |
| clone set: | RP11-1093B19 | RP11-384D8 | RP11-662L9 | |
| clone set: | RP11-1093B19 | RP11-533M23 | RP11-644F13 | |
| clone set: | RP11-1093B19 | RP11-533M23 | RP11-264K18 | |
| clone set: | RP11-1093B19 | RP11-533M23 | RP11-416O8 | |
| clone set: | RP11-1093B19 | RP11-533M23 | RP11-90M19 | |
| clone set: | RP11-1093B19 | RP11-533M23 | RP11-662L9 | |
| clone set: | RP11-813D1 | RP11-430B3 | RP11-644F13 | |
| clone set: | RP11-813D1 | RP11-430B3 | RP11-264K18 | |
| clone set: | RP11-813D1 | RP11-430B3 | RP11-416O8 | |
| clone set: | RP11-813D1 | RP11-430B3 | RP11-90M19 | |
| clone set: | RP11-813D1 | RP11-430B3 | RP11-662L9 | |
| clone set: | RP11-813D1 | RP11-98E9 | RP11-644F13 | |
| clone set: | RP11-813D1 | RP11-98E9 | RP11-264K18 | |
| clone set: | RP11-813D1 | RP11-98E9 | RP11-416O8 | |
| clone set: | RP11-813D1 | RP11-98E9 | RP11-90M19 | |
| clone set: | RP11-813D1 | RP11-98E9 | RP11-662L9 | |
| clone set: | RP11-813D1 | RP11-58M12 | RP11-644F13 | |
| clone set: | RP11-813D1 | RP11-58M12 | RP11-264K18 | |
| clone set: | RP11-813D1 | RP11-58M12 | RP11-416O8 | |
| clone set: | RP11-813D1 | RP11-58M12 | RP11-90M19 | |
| clone set: | RP11-813D1 | RP11-58M12 | RP11-662L9 | |
| clone set: | RP11-813D1 | RP11-384D8 | RP11-644F13 | |
| clone set: | RP11-813D1 | RP11-384D8 | RP11-264K18 | |
| clone set: | RP11-813D1 | RP11-384D8 | RP11-416O8 | |
| clone set: | RP11-813D1 | RP11-384D8 | RP11-90M19 | |
| clone set: | RP11-813D1 | RP11-384D8 | RP11-662L9 | |
| clone set: | RP11-813D1 | RP11-533M23 | RP11-644F13 | |
| clone set: | RP11-813D1 | RP11-533M23 | RP11-264K18 | |
| clone set: | RP11-813D1 | RP11-533M23 | RP11-416O8 | |
| clone set: | RP11-813D1 | RP11-533M23 | RP11-90M19 | |
| clone set: | RP11-813D1 | RP11-533M23 | RP11-662L9 | |
| clone set: | RP11-432N19 | RP11-430B3 | RP11-644F13 | |
| clone set: | RP11-432N19 | RP11-430B3 | RP11-264K18 | |
| clone set: | RP11-432N19 | RP11-430B3 | RP11-416O8 | |
| clone set: | RP11-432N19 | RP11-430B3 | RP11-90M19 | |
| clone set: | RP11-432N19 | RP11-430B3 | RP11-662L9 | |
| clone set: | RP11-432N19 | RP11-98E9 | RP11-644F13 | |
| clone set: | RP11-432N19 | RP11-98E9 | RP11-264K18 | |
| clone set: | RP11-432N19 | RP11-98E9 | RP11-416O8 | |
| clone set: | RP11-432N19 | RP11-98E9 | RP11-90M19 | |
| clone set: | RP11-432N19 | RP11-98E9 | RP11-662L9 | |
| clone set: | RP11-432N19 | RP11-58M12 | RP11-644F13 | |
| clone set: | RP11-432N19 | RP11-58M12 | RP11-264K18 | |
| clone set: | RP11-432N19 | RP11-58M12 | RP11-416O8 | |
| clone set: | RP11-432N19 | RP11-58M12 | RP11-90M19 | |
| clone set: | RP11-432N19 | RP11-58M12 | RP11-662L9 | |
| clone set: | RP11-432N19 | RP11-384D8 | RP11-644F13 | |
| clone set: | RP11-432N19 | RP11-384D8 | RP11-264K18 | |
| clone set: | RP11-432N19 | RP11-384D8 | RP11-416O8 | |
| clone set: | RP11-432N19 | RP11-384D8 | RP11-90M19 | |
| clone set: | RP11-432N19 | RP11-384D8 | RP11-662L9 | |
| clone set: | RP11-432N19 | RP11-533M23 | RP11-644F13 | |
| clone set: | RP11-432N19 | RP11-533M23 | RP11-264K18 | |
| clone set: | RP11-432N19 | RP11-533M23 | RP11-416O8 | |
| clone set: | RP11-432N19 | RP11-533M23 | RP11-90M19 | |
| clone set: | RP11-432N19 | RP11-533M23 | RP11-662L9 | |
| clone set: | RP11-719G24 | RP11-430B3 | RP11-533M23 | RP11-90M19 |
| clone set: | RP11-719G24 | RP11-430B3 | RP11-533M23 | RP11-662L9 |
| clone set: | RP11-719G24 | RP11-430B3 | RP11-644F13 | RP11-90M19 |
| clone set: | RP11-719G24 | RP11-430B3 | RP11-644F13 | RP11-662L9 |
| clone set: | RP11-719G24 | RP11-430B3 | RP11-264K18 | RP11-90M19 |
| clone set: | RP11-719G24 | RP11-430B3 | RP11-264K18 | RP11-662L9 |
| clone set: | RP11-719G24 | RP11-430B3 | RP11-416O8 | RP11-90M19 |
| clone set: | RP11-719G24 | RP11-430B3 | RP11-416O8 | RP11-662L9 |
| clone set: | RP11-719G24 | RP11-98E9 | RP11-533M23 | RP11-90M19 |
| clone set: | RP11-719G24 | RP11-98E9 | RP11-533M23 | RP11-662L9 |
| clone set: | RP11-719G24 | RP11-98E9 | RP11-644F13 | RP11-90M19 |
| clone set: | RP11-719G24 | RP11-98E9 | RP11-644F13 | RP11-662L9 |
| clone set: | RP11-719G24 | RP11-98E9 | RP11-264K18 | RP11-90M19 |
| clone set: | RP11-719G24 | RP11-98E9 | RP11-264K18 | RP11-662L9 |
| clone set: | RP11-719G24 | RP11-98E9 | RP11-416O8 | RP11-90M19 |
| clone set: | RP11-719G24 | RP11-98E9 | RP11-416O8 | RP11-662L9 |
| clone set: | RP11-719G24 | RP11-58M12 | RP11-533M23 | RP11-90M19 |
| clone set: | RP11-719G24 | RP11-58M12 | RP11-533M23 | RP11-662L9 |
| clone set: | RP11-719G24 | RP11-58M12 | RP11-644F13 | RP11-90M19 |
| clone set: | RP11-719G24 | RP11-58M12 | RP11-644F13 | RP11-662L9 |
| clone set: | RP11-719G24 | RP11-58M12 | RP11-264K18 | RP11-90M19 |
| clone set: | RP11-719G24 | RP11-58M12 | RP11-264K18 | RP11-662L9 |
| clone set: | RP11-719G24 | RP11-58M12 | RP11-416O8 | RP11-90M19 |
| clone set: | RP11-719G24 | RP11-58M12 | RP11-416O8 | RP11-662L9 |
| clone set: | RP11-719G24 | RP11-384D8 | RP11-533M23 | RP11-90M19 |
| clone set: | RP11-719G24 | RP11-384D8 | RP11-533M23 | RP11-662L9 |
| clone set: | RP11-719G24 | RP11-384D8 | RP11-644F13 | RP11-90M19 |
| clone set: | RP11-719G24 | RP11-384D8 | RP11-644F13 | RP11-662L9 |
| clone set: | RP11-719G24 | RP11-384D8 | RP11-264K18 | RP11-90M19 |
| clone set: | RP11-719G24 | RP11-384D8 | RP11-264K18 | RP11-662L9 |
| clone set: | RP11-719G24 | RP11-384D8 | RP11-416O8 | RP11-90M19 |
| clone set: | RP11-719G24 | RP11-384D8 | RP11-416O8 | RP11-662L9 |
| clone set: | RP11-1093B19 | RP11-430B3 | RP11-533M23 | RP11-90M19 |
| clone set: | RP11-1093B19 | RP11-430B3 | RP11-533M23 | RP11-662L9 |
| clone set: | RP11-1093B19 | RP11-430B3 | RP11-644F13 | RP11-90M19 |
| clone set: | RP11-1093B19 | RP11-430B3 | RP11-644F13 | RP11-662L9 |
| clone set: | RP11-1093B19 | RP11-430B3 | RP11-264K18 | RP11-90M19 |
| clone set: | RP11-1093B19 | RP11-430B3 | RP11-264K18 | RP11-662L9 |
| clone set: | RP11-1093B19 | RP11-430B3 | RP11-416O8 | RP11-90M19 |
| clone set: | RP11-1093B19 | RP11-430B3 | RP11-416O8 | RP11-662L9 |
| clone set: | RP11-1093B19 | RP11-98E9 | RP11-533M23 | RP11-90M19 |
| clone set: | RP11-1093B19 | RP11-98E9 | RP11-533M23 | RP11-662L9 |
| clone set: | RP11-1093B19 | RP11-98E9 | RP11-644F13 | RP11-90M19 |
| clone set: | RP11-1093B19 | RP11-98E9 | RP11-644F13 | RP11-662L9 |
| clone set: | RP11-1093B19 | RP11-98E9 | RP11-264K18 | RP11-90M19 |
| clone set: | RP11-1093B19 | RP11-98E9 | RP11-264K18 | RP11-662L9 |
| clone set: | RP11-1093B19 | RP11-98E9 | RP11-416O8 | RP11-90M19 |
| clone set: | RP11-1093B19 | RP11-98E9 | RP11-416O8 | RP11-662L9 |
| clone set: | RP11-1093B19 | RP11-58M12 | RP11-533M23 | RP11-90M19 |
| clone set: | RP11-1093B19 | RP11-58M12 | RP11-533M23 | RP11-662L9 |
| clone set: | RP11-1093B19 | RP11-58M12 | RP11-644F13 | RP11-90M19 |
| clone set: | RP11-1093B19 | RP11-58M12 | RP11-644F13 | RP11-662L9 |
| clone set: | RP11-1093B19 | RP11-58M12 | RP11-264K18 | RP11-90M19 |
| clone set: | RP11-1093B19 | RP11-58M12 | RP11-264K18 | RP11-662L9 |
| clone set: | RP11-1093B19 | RP11-58M12 | RP11-416O8 | RP11-90M19 |
| clone set: | RP11-1093B19 | RP11-58M12 | RP11-416O8 | RP11-662L9 |
| clone set: | RP11-1093B19 | RP11-384D8 | RP11-533M23 | RP11-90M19 |
| clone set: | RP11-1093B19 | RP11-384D8 | RP11-533M23 | RP11-662L9 |
| clone set: | RP11-1093B19 | RP11-384D8 | RP11-644F13 | RP11-90M19 |
| clone set: | RP11-1093B19 | RP11-384D8 | RP11-644F13 | RP11-662L9 |
| clone set: | RP11-1093B19 | RP11-384D8 | RP11-264K18 | RP11-90M19 |
| clone set: | RP11-1093B19 | RP11-384D8 | RP11-264K18 | RP11-662L9 |
| clone set: | RP11-1093B19 | RP11-384D8 | RP11-416O8 | RP11-90M19 |
| clone set: | RP11-1093B19 | RP11-384D8 | RP11-416O8 | RP11-662L9 |
| clone set: | RP11-813D1 | RP11-430B3 | RP11-533M23 | RP11-90M19 |
| clone set: | RP11-813D1 | RP11-430B3 | RP11-533M23 | RP11-662L9 |
| clone set: | RP11-813D1 | RP11-430B3 | RP11-644F13 | RP11-90M19 |
| clone set: | RP11-813D1 | RP11-430B3 | RP11-644F13 | RP11-662L9 |
| clone set: | RP11-813D1 | RP11-430B3 | RP11-264K18 | RP11-90M19 |
| clone set: | RP11-813D1 | RP11-430B3 | RP11-264K18 | RP11-662L9 |
| clone set: | RP11-813D1 | RP11-430B3 | RP11-416O8 | RP11-90M19 |
| clone set: | RP11-813D1 | RP11-430B3 | RP11-416O8 | RP11-662L9 |
| clone set: | RP11-813D1 | RP11-98E9 | RP11-533M23 | RP11-90M19 |
| clone set: | RP11-813D1 | RP11-98E9 | RP11-533M23 | RP11-662L9 |
| clone set: | RP11-813D1 | RP11-98E9 | RP11-644F13 | RP11-90M19 |
| clone set: | RP11-813D1 | RP11-98E9 | RP11-644F13 | RP11-662L9 |
| clone set: | RP11-813D1 | RP11-98E9 | RP11-264K18 | RP11-90M19 |
| clone set: | RP11-813D1 | RP11-98E9 | RP11-264K18 | RP11-662L9 |
| clone set: | RP11-813D1 | RP11-98E9 | RP11-416O8 | RP11-90M19 |
| clone set: | RP11-813D1 | RP11-98E9 | RP11-416O8 | RP11-662L9 |
| clone set: | RP11-813D1 | RP11-58M12 | RP11-533M23 | RP11-90M19 |
| clone set: | RP11-813D1 | RP11-58M12 | RP11-533M23 | RP11-662L9 |
| clone set: | RP11-813D1 | RP11-58M12 | RP11-644F13 | RP11-90M19 |
| clone set: | RP11-813D1 | RP11-58M12 | RP11-644F13 | RP11-662L9 |
| clone set: | RP11-813D1 | RP11-58M12 | RP11-264K18 | RP11-90M19 |
| clone set: | RP11-813D1 | RP11-58M12 | RP11-264K18 | RP11-662L9 |
| clone set: | RP11-813D1 | RP11-58M12 | RP11-416O8 | RP11-90M19 |
| clone set: | RP11-813D1 | RP11-58M12 | RP11-416O8 | RP11-662L9 |
| clone set: | RP11-813D1 | RP11-384D8 | RP11-533M23 | RP11-90M19 |
| clone set: | RP11-813D1 | RP11-384D8 | RP11-533M23 | RP11-662L9 |
| clone set: | RP11-813D1 | RP11-384D8 | RP11-644F13 | RP11-90M19 |
| clone set: | RP11-813D1 | RP11-384D8 | RP11-644F13 | RP11-662L9 |
| clone set: | RP11-813D1 | RP11-384D8 | RP11-264K18 | RP11-90M19 |
| clone set: | RP11-813D1 | RP11-384D8 | RP11-264K18 | RP11-662L9 |
| clone set: | RP11-813D1 | RP11-384D8 | RP11-416O8 | RP11-90M19 |
| clone set: | RP11-813D1 | RP11-384D8 | RP11-416O8 | RP11-662L9 |
| clone set: | RP11-432N19 | RP11-430B3 | RP11-533M23 | RP11-90M19 |
| clone set: | RP11-432N19 | RP11-430B3 | RP11-533M23 | RP11-662L9 |
| clone set: | RP11-432N19 | RP11-430B3 | RP11-644F13 | RP11-90M19 |
| clone set: | RP11-432N19 | RP11-430B3 | RP11-644F13 | RP11-662L9 |
| clone set: | RP11-432N19 | RP11-430B3 | RP11-264K18 | RP11-90M19 |
| clone set: | RP11-432N19 | RP11-430B3 | RP11-264K18 | RP11-662L9 |
| clone set: | RP11-432N19 | RP11-430B3 | RP11-416O8 | RP11-90M19 |
| clone set: | RP11-432N19 | RP11-430B3 | RP11-416O8 | RP11-662L9 |
| clone set: | RP11-432N19 | RP11-98E9 | RP11-533M23 | RP11-90M19 |
| clone set: | RP11-432N19 | RP11-98E9 | RP11-533M23 | RP11-662L9 |
| clone set: | RP11-432N19 | RP11-98E9 | RP11-644F13 | RP11-90M19 |
| clone set: | RP11-432N19 | RP11-98E9 | RP11-644F13 | RP11-662L9 |
| clone set: | RP11-432N19 | RP11-98E9 | RP11-264K18 | RP11-90M19 |
| clone set: | RP11-432N19 | RP11-98E9 | RP11-264K18 | RP11-662L9 |
| clone set: | RP11-432N19 | RP11-98E9 | RP11-416O8 | RP11-90M19 |
| clone set: | RP11-432N19 | RP11-98E9 | RP11-416O8 | RP11-662L9 |
| clone set: | RP11-432N19 | RP11-58M12 | RP11-533M23 | RP11-90M19 |
| clone set: | RP11-432N19 | RP11-58M12 | RP11-533M23 | RP11-662L9 |
| clone set: | RP11-432N19 | RP11-58M12 | RP11-644F13 | RP11-90M19 |
| clone set: | RP11-432N19 | RP11-58M12 | RP11-644F13 | RP11-662L9 |
| clone set: | RP11-432N19 | RP11-58M12 | RP11-264K18 | RP11-90M19 |
| clone set: | RP11-432N19 | RP11-58M12 | RP11-264K18 | RP11-662L9 |
| clone set: | RP11-432N19 | RP11-58M12 | RP11-416O8 | RP11-90M19 |
| clone set: | RP11-432N19 | RP11-58M12 | RP11-416O8 | RP11-662L9 |
| clone set: | RP11-432N19 | RP11-384D8 | RP11-533M23 | RP11-90M19 |
| clone set: | RP11-432N19 | RP11-384D8 | RP11-533M23 | RP11-662L9 |
| clone set: | RP11-432N19 | RP11-384D8 | RP11-644F13 | RP11-90M19 |
| clone set: | RP11-432N19 | RP11-384D8 | RP11-644F13 | RP11-662L9 |
| clone set: | RP11-432N19 | RP11-384D8 | RP11-264K18 | RP11-90M19 |
| clone set: | RP11-432N19 | RP11-384D8 | RP11-264K18 | RP11-662L9 |
| clone set: | RP11-432N19 | RP11-384D8 | RP11-416O8 | RP11-90M19 |
| clone set: | RP11-432N19 | RP11-384D8 | RP11-416O8 | RP11-662L9 |
| clone set: | RP11-719G24 | RP11-813D1 | RP11-98E9 | RP11-533M23 |
| clone set: | RP11-719G24 | RP11-813D1 | RP11-98E9 | RP11-533M23 |
| clone set: | RP11-719G24 | RP11-813D1 | RP11-98E9 | RP11-533M23 |
| clone set: | RP11-719G24 | RP11-813D1 | RP11-98E9 | RP11-644F13 |
| clone set: | RP11-719G24 | RP11-813D1 | RP11-98E9 | RP11-644F13 |
| clone set: | RP11-719G24 | RP11-813D1 | RP11-98E9 | RP11-644F13 |
| clone set: | RP11-719G24 | RP11-813D1 | RP11-98E9 | RP11-264K18 |
| clone set: | RP11-719G24 | RP11-813D1 | RP11-98E9 | RP11-264K18 |
| clone set: | RP11-719G24 | RP11-813D1 | RP11-98E9 | RP11-264K18 |
| clone set: | RP11-719G24 | RP11-813D1 | RP11-58M12 | RP11-533M23 |
| clone set: | RP11-719G24 | RP11-813D1 | RP11-58M12 | RP11-533M23 |
| clone set: | RP11-719G24 | RP11-813D1 | RP11-58M12 | RP11-533M23 |
| clone set: | RP11-719G24 | RP11-813D1 | RP11-58M12 | RP11-644F13 |
| clone set: | RP11-719G24 | RP11-813D1 | RP11-58M12 | RP11-644F13 |
| clone set: | RP11-719G24 | RP11-813D1 | RP11-58M12 | RP11-644F13 |
| clone set: | RP11-719G24 | RP11-813D1 | RP11-58M12 | RP11-264K18 |
| clone set: | RP11-719G24 | RP11-813D1 | RP11-58M12 | RP11-264K18 |
| clone set: | RP11-719G24 | RP11-813D1 | RP11-58M12 | RP11-264K18 |
| clone set: | RP11-719G24 | RP11-813D1 | RP11-384D8 | RP11-533M23 |
| clone set: | RP11-719G24 | RP11-813D1 | RP11-384D8 | RP11-533M23 |
| clone set: | RP11-719G24 | RP11-813D1 | RP11-384D8 | RP11-533M23 |
| clone set: | RP11-719G24 | RP11-813D1 | RP11-384D8 | RP11-644F13 |
| clone set: | RP11-719G24 | RP11-813D1 | RP11-384D8 | RP11-644F13 |
| clone set: | RP11-719G24 | RP11-813D1 | RP11-384D8 | RP11-644F13 |
| clone set: | RP11-719G24 | RP11-813D1 | RP11-384D8 | RP11-264K18 |
| clone set: | RP11-719G24 | RP11-813D1 | RP11-384D8 | RP11-264K18 |
| clone set: | RP11-719G24 | RP11-813D1 | RP11-384D8 | RP11-264K18 |
| clone set: | RP11-719G24 | RP11-432N19 | RP11-98E9 | RP11-533M23 |
| clone set: | RP11-719G24 | RP11-432N19 | RP11-98E9 | RP11-533M23 |
| clone set: | RP11-719G24 | RP11-432N19 | RP11-98E9 | RP11-533M23 |
| clone set: | RP11-719G24 | RP11-432N19 | RP11-98E9 | RP11-644F13 |
| clone set: | RP11-719G24 | RP11-432N19 | RP11-98E9 | RP11-644F13 |
| clone set: | RP11-719G24 | RP11-432N19 | RP11-98E9 | RP11-644F13 |
| clone set: | RP11-719G24 | RP11-432N19 | RP11-98E9 | RP11-264K18 |
| clone set: | RP11-719G24 | RP11-432N19 | RP11-98E9 | RP11-264K18 |
| clone set: | RP11-719G24 | RP11-432N19 | RP11-98E9 | RP11-264K18 |
| clone set: | RP11-719G24 | RP11-432N19 | RP11-58M12 | RP11-533M23 |
| clone set: | RP11-719G24 | RP11-432N19 | RP11-58M12 | RP11-533M23 |
| clone set: | RP11-719G24 | RP11-432N19 | RP11-58M12 | RP11-533M23 |
| clone set: | RP11-719G24 | RP11-432N19 | RP11-58M12 | RP11-644F13 |
| clone set: | RP11-719G24 | RP11-432N19 | RP11-58M12 | RP11-644F13 |
| clone set: | RP11-719G24 | RP11-432N19 | RP11-58M12 | RP11-644F13 |
| clone set: | RP11-719G24 | RP11-432N19 | RP11-58M12 | RP11-264K18 |
| clone set: | RP11-719G24 | RP11-432N19 | RP11-58M12 | RP11-264K18 |
| clone set: | RP11-719G24 | RP11-432N19 | RP11-58M12 | RP11-264K18 |
| clone set: | RP11-719G24 | RP11-432N19 | RP11-384D8 | RP11-533M23 |
| clone set: | RP11-719G24 | RP11-432N19 | RP11-384D8 | RP11-533M23 |
| clone set: | RP11-719G24 | RP11-432N19 | RP11-384D8 | RP11-533M23 |
| clone set: | RP11-719G24 | RP11-432N19 | RP11-384D8 | RP11-644F13 |
| clone set: | RP11-719G24 | RP11-432N19 | RP11-384D8 | RP11-644F13 |
| clone set: | RP11-719G24 | RP11-432N19 | RP11-384D8 | RP11-644F13 |
| clone set: | RP11-719G24 | RP11-432N19 | RP11-384D8 | RP11-264K18 |
| clone set: | RP11-719G24 | RP11-432N19 | RP11-384D8 | RP11-264K18 |
| clone set: | RP11-719G24 | RP11-432N19 | RP11-384D8 | RP11-264K18 |
| clone set: | RP11-719G24 | RP11-430B3 | RP11-98E9 | RP11-533M23 |
| clone set: | RP11-719G24 | RP11-430B3 | RP11-98E9 | RP11-533M23 |
| clone set: | RP11-719G24 | RP11-430B3 | RP11-98E9 | RP11-533M23 |
| clone set: | RP11-719G24 | RP11-430B3 | RP11-98E9 | RP11-644F13 |
| clone set: | RP11-719G24 | RP11-430B3 | RP11-98E9 | RP11-644F13 |
| clone set: | RP11-719G24 | RP11-430B3 | RP11-98E9 | RP11-644F13 |
| clone set: | RP11-719G24 | RP11-430B3 | RP11-98E9 | RP11-264K18 |
| clone set: | RP11-719G24 | RP11-430B3 | RP11-98E9 | RP11-264K18 |
| clone set: | RP11-719G24 | RP11-430B3 | RP11-98E9 | RP11-264K18 |
| clone set: | RP11-719G24 | RP11-430B3 | RP11-58M12 | RP11-533M23 |
| clone set: | RP11-719G24 | RP11-430B3 | RP11-58M12 | RP11-533M23 |
| clone set: | RP11-719G24 | RP11-430B3 | RP11-58M12 | RP11-533M23 |
| clone set: | RP11-719G24 | RP11-430B3 | RP11-58M12 | RP11-644F13 |
| clone set: | RP11-719G24 | RP11-430B3 | RP11-58M12 | RP11-644F13 |
| clone set: | RP11-719G24 | RP11-430B3 | RP11-58M12 | RP11-644F13 |
| clone set: | RP11-719G24 | RP11-430B3 | RP11-58M12 | RP11-264K18 |
| clone set: | RP11-719G24 | RP11-430B3 | RP11-58M12 | RP11-264K18 |
| clone set: | RP11-719G24 | RP11-430B3 | RP11-58M12 | RP11-264K18 |
| clone set: | RP11-719G24 | RP11-430B3 | RP11-384D8 | RP11-533M23 |
| clone set: | RP11-719G24 | RP11-430B3 | RP11-384D8 | RP11-533M23 |
| clone set: | RP11-719G24 | RP11-430B3 | RP11-384D8 | RP11-533M23 |
| clone set: | RP11-719G24 | RP11-430B3 | RP11-384D8 | RP11-644F13 |
| clone set: | RP11-719G24 | RP11-430B3 | RP11-384D8 | RP11-644F13 |
| clone set: | RP11-719G24 | RP11-430B3 | RP11-384D8 | RP11-644F13 |
| clone set: | RP11-719G24 | RP11-430B3 | RP11-384D8 | RP11-264K18 |
| clone set: | RP11-719G24 | RP11-430B3 | RP11-384D8 | RP11-264K18 |
| clone set: | RP11-719G24 | RP11-430B3 | RP11-384D8 | RP11-264K18 |
| clone set: | RP11-1093B19 | RP11-813D1 | RP11-98E9 | RP11-533M23 |
| clone set: | RP11-1093B19 | RP11-813D1 | RP11-98E9 | RP11-533M23 |
| clone set: | RP11-1093B19 | RP11-813D1 | RP11-98E9 | RP11-533M23 |
| clone set: | RP11-1093B19 | RP11-813D1 | RP11-98E9 | RP11-644F13 |
| clone set: | RP11-1093B19 | RP11-813D1 | RP11-98E9 | RP11-644F13 |
| clone set: | RP11-1093B19 | RP11-813D1 | RP11-98E9 | RP11-644F13 |
| clone set: | RP11-1093B19 | RP11-813D1 | RP11-98E9 | RP11-264K18 |
| clone set: | RP11-1093B19 | RP11-813D1 | RP11-98E9 | RP11-264K18 |
| clone set: | RP11-1093B19 | RP11-813D1 | RP11-98E9 | RP11-264K18 |
| clone set: | RP11-1093B19 | RP11-813D1 | RP11-58M12 | RP11-533M23 |
| clone set: | RP11-1093B19 | RP11-813D1 | RP11-58M12 | RP11-533M23 |
| clone set: | RP11-1093B19 | RP11-813D1 | RP11-58M12 | RP11-533M23 |
| clone set: | RP11-1093B19 | RP11-813D1 | RP11-58M12 | RP11-644F13 |
| clone set: | RP11-1093B19 | RP11-813D1 | RP11-58M12 | RP11-644F13 |
| clone set: | RP11-1093B19 | RP11-813D1 | RP11-58M12 | RP11-644F13 |
| clone set: | RP11-1093B19 | RP11-813D1 | RP11-58M12 | RP11-264K18 |
| clone set: | RP11-1093B19 | RP11-813D1 | RP11-58M12 | RP11-264K18 |
| clone set: | RP11-1093B19 | RP11-813D1 | RP11-58M12 | RP11-264K18 |
| clone set: | RP11-1093B19 | RP11-813D1 | RP11-384D8 | RP11-533M23 |
| clone set: | RP11-1093B19 | RP11-813D1 | RP11-384D8 | RP11-533M23 |
| clone set: | RP11-1093B19 | RP11-813D1 | RP11-384D8 | RP11-533M23 |
| clone set: | RP11-1093B19 | RP11-813D1 | RP11-384D8 | RP11-644F13 |
| clone set: | RP11-1093B19 | RP11-813D1 | RP11-384D8 | RP11-644F13 |
| clone set: | RP11-1093B19 | RP11-813D1 | RP11-384D8 | RP11-644F13 |
| clone set: | RP11-1093B19 | RP11-813D1 | RP11-384D8 | RP11-264K18 |
| clone set: | RP11-1093B19 | RP11-813D1 | RP11-384D8 | RP11-264K18 |
| clone set: | RP11-1093B19 | RP11-813D1 | RP11-384D8 | RP11-264K18 |
| clone set: | RP11-1093B19 | RP11-432N19 | RP11-98E9 | RP11-533M23 |
| clone set: | RP11-1093B19 | RP11-432N19 | RP11-98E9 | RP11-533M23 |
| clone set: | RP11-1093B19 | RP11-432N19 | RP11-98E9 | RP11-533M23 |
| clone set: | RP11-1093B19 | RP11-432N19 | RP11-98E9 | RP11-644F13 |
| clone set: | RP11-1093B19 | RP11-432N19 | RP11-98E9 | RP11-644F13 |
| clone set: | RP11-1093B19 | RP11-432N19 | RP11-98E9 | RP11-644F13 |
| clone set: | RP11-1093B19 | RP11-432N19 | RP11-98E9 | RP11-264K18 |
| clone set: | RP11-1093B19 | RP11-432N19 | RP11-98E9 | RP11-264K18 |
| clone set: | RP11-1093B19 | RP11-432N19 | RP11-98E9 | RP11-264K18 |
| clone set: | RP11-1093B19 | RP11-432N19 | RP11-58M12 | RP11-533M23 |
| clone set: | RP11-1093B19 | RP11-432N19 | RP11-58M12 | RP11-533M23 |
| clone set: | RP11-1093B19 | RP11-432N19 | RP11-58M12 | RP11-533M23 |
| clone set: | RP11-1093B19 | RP11-432N19 | RP11-58M12 | RP11-644F13 |
| clone set: | RP11-1093B19 | RP11-432N19 | RP11-58M12 | RP11-644F13 |
| clone set: | RP11-1093B19 | RP11-432N19 | RP11-58M12 | RP11-644F13 |
| clone set: | RP11-1093B19 | RP11-432N19 | RP11-58M12 | RP11-264K18 |
| clone set: | RP11-1093B19 | RP11-432N19 | RP11-58M12 | RP11-264K18 |
| clone set: | RP11-1093B19 | RP11-432N19 | RP11-58M12 | RP11-264K18 |
| clone set: | RP11-1093B19 | RP11-432N19 | RP11-384D8 | RP11-533M23 |
| clone set: | RP11-1093B19 | RP11-432N19 | RP11-384D8 | RP11-533M23 |
| clone set: | RP11-1093B19 | RP11-432N19 | RP11-384D8 | RP11-533M23 |
| clone set: | RP11-1093B19 | RP11-432N19 | RP11-384D8 | RP11-644F13 |
| clone set: | RP11-1093B19 | RP11-432N19 | RP11-384D8 | RP11-644F13 |
| clone set: | RP11-1093B19 | RP11-432N19 | RP11-384D8 | RP11-644F13 |
| clone set: | RP11-1093B19 | RP11-432N19 | RP11-384D8 | RP11-264K18 |
| clone set: | RP11-1093B19 | RP11-432N19 | RP11-384D8 | RP11-264K18 |
| clone set: | RP11-1093B19 | RP11-432N19 | RP11-384D8 | RP11-264K18 |
| clone set: | RP11-1093B19 | RP11-430B3 | RP11-98E9 | RP11-533M23 |
| clone set: | RP11-1093B19 | RP11-430B3 | RP11-98E9 | RP11-533M23 |
| clone set: | RP11-1093B19 | RP11-430B3 | RP11-98E9 | RP11-533M23 |
| clone set: | RP11-1093B19 | RP11-430B3 | RP11-98E9 | RP11-644F13 |
| clone set: | RP11-1093B19 | RP11-430B3 | RP11-98E9 | RP11-644F13 |
| clone set: | RP11-1093B19 | RP11-430B3 | RP11-98E9 | RP11-644F13 |
| clone set: | RP11-1093B19 | RP11-430B3 | RP11-98E9 | RP11-264K18 |
| clone set: | RP11-1093B19 | RP11-430B3 | RP11-98E9 | RP11-264K18 |
| clone set: | RP11-1093B19 | RP11-430B3 | RP11-98E9 | RP11-264K18 |
| clone set: | RP11-1093B19 | RP11-430B3 | RP11-58M12 | RP11-533M23 |
| clone set: | RP11-1093B19 | RP11-430B3 | RP11-58M12 | RP11-533M23 |
| clone set: | RP11-1093B19 | RP11-430B3 | RP11-58M12 | RP11-533M23 |
| clone set: | RP11-1093B19 | RP11-430B3 | RP11-58M12 | RP11-644F13 |
| clone set: | RP11-1093B19 | RP11-430B3 | RP11-58M12 | RP11-644F13 |
| clone set: | RP11-1093B19 | RP11-430B3 | RP11-58M12 | RP11-644F13 |
| clone set: | RP11-1093B19 | RP11-430B3 | RP11-58M12 | RP11-264K18 |
| clone set: | RP11-1093B19 | RP11-430B3 | RP11-58M12 | RP11-264K18 |
| clone set: | RP11-1093B19 | RP11-430B3 | RP11-58M12 | RP11-264K18 |
| clone set: | RP11-1093B19 | RP11-430B3 | RP11-384D8 | RP11-533M23 |
| clone set: | RP11-1093B19 | RP11-430B3 | RP11-384D8 | RP11-533M23 |
| clone set: | RP11-1093B19 | RP11-430B3 | RP11-384D8 | RP11-533M23 |
| clone set: | RP11-1093B19 | RP11-430B3 | RP11-384D8 | RP11-644F13 |
| clone set: | RP11-1093B19 | RP11-430B3 | RP11-384D8 | RP11-644F13 |
| clone set: | RP11-1093B19 | RP11-430B3 | RP11-384D8 | RP11-644F13 |
| clone set: | RP11-1093B19 | RP11-430B3 | RP11-384D8 | RP11-264K18 |
| clone set: | RP11-1093B19 | RP11-430B3 | RP11-384D8 | RP11-264K18 |
| clone set: | RP11-1093B19 | RP11-430B3 | RP11-384D8 | RP11-264K18 |
| clone set: | RP11-416O6 | RP11-662L9 | RP11-98E9 | RP11-58M12 |
| clone set: | RP11-813D1 | RP11-813D1 | RP11-98E9 | RP11-533M23 |
| clone set: | RP11-813D1 | RP11-813D1 | RP11-98E9 | RP11-533M23 |
| clone set: | RP11-813D1 | RP11-813D1 | RP11-98E9 | RP11-533M23 |
| clone set: | RP11-813D1 | RP11-813D1 | RP11-98E9 | RP11-644F13 |
| clone set: | RP11-813D1 | RP11-813D1 | RP11-98E9 | RP11-644F13 |
| clone set: | RP11-813D1 | RP11-813D1 | RP11-98E9 | RP11-644F13 |
| clone set: | RP11-813D1 | RP11-813D1 | RP11-98E9 | RP11-264K18 |
| clone set: | RP11-813D1 | RP11-813D1 | RP11-98E9 | RP11-264K18 |
| clone set: | RP11-813D1 | RP11-813D1 | RP11-98E9 | RP11-264K18 |
| clone set: | RP11-813D1 | RP11-813D1 | RP11-58M12 | RP11-533M23 |
| clone set: | RP11-813D1 | RP11-813D1 | RP11-58M12 | RP11-533M23 |
| clone set: | RP11-813D1 | RP11-813D1 | RP11-58M12 | RP11-533M23 |
| clone set: | RP11-813D1 | RP11-813D1 | RP11-58M12 | RP11-644F13 |
| clone set: | RP11-813D1 | RP11-813D1 | RP11-58M12 | RP11-644F13 |
| clone set: | RP11-813D1 | RP11-813D1 | RP11-58M12 | RP11-644F13 |
| clone set: | RP11-813D1 | RP11-813D1 | RP11-58M12 | RP11-264K18 |
| clone set: | RP11-813D1 | RP11-813D1 | RP11-58M12 | RP11-264K18 |
| clone set: | RP11-813D1 | RP11-813D1 | RP11-58M12 | RP11-264K18 |
| clone set: | RP11-813D1 | RP11-813D1 | RP11-384D8 | RP11-533M23 |
| clone set: | RP11-813D1 | RP11-813D1 | RP11-384D8 | RP11-533M23 |
| clone set: | RP11-813D1 | RP11-813D1 | RP11-384D8 | RP11-533M23 |
| clone set: | RP11-813D1 | RP11-813D1 | RP11-384D8 | RP11-644F13 |
| clone set: | RP11-813D1 | RP11-813D1 | RP11-384D8 | RP11-644F13 |
| clone set: | RP11-813D1 | RP11-813D1 | RP11-384D8 | RP11-644F13 |
| clone set: | RP11-813D1 | RP11-813D1 | RP11-384D8 | RP11-264K18 |
| clone set: | RP11-813D1 | RP11-813D1 | RP11-384D8 | RP11-264K18 |
| clone set: | RP11-813D1 | RP11-813D1 | RP11-384D8 | RP11-264K18 |
| clone set: | RP11-813D1 | RP11-432N19 | RP11-98E9 | RP11-533M23 |
| clone set: | RP11-813D1 | RP11-432N19 | RP11-98E9 | RP11-533M23 |
| clone set: | RP11-813D1 | RP11-432N19 | RP11-98E9 | RP11-533M23 |
| clone set: | RP11-813D1 | RP11-432N19 | RP11-98E9 | RP11-644F13 |
| clone set: | RP11-813D1 | RP11-432N19 | RP11-98E9 | RP11-644F13 |
| clone set: | RP11-813D1 | RP11-432N19 | RP11-98E9 | RP11-644F13 |
| clone set: | RP11-813D1 | RP11-432N19 | RP11-98E9 | RP11-264K18 |
| clone set: | RP11-813D1 | RP11-432N19 | RP11-98E9 | RP11-264K18 |
| clone set: | RP11-813D1 | RP11-432N19 | RP11-98E9 | RP11-264K18 |
| clone set: | RP11-813D1 | RP11-432N19 | RP11-58M12 | RP11-533M23 |
| clone set: | RP11-813D1 | RP11-432N19 | RP11-58M12 | RP11-533M23 |
| clone set: | RP11-813D1 | RP11-432N19 | RP11-58M12 | RP11-533M23 |
| clone set: | RP11-813D1 | RP11-432N19 | RP11-58M12 | RP11-644F13 |
| clone set: | RP11-813D1 | RP11-432N19 | RP11-58M12 | RP11-644F13 |
| clone set: | RP11-813D1 | RP11-432N19 | RP11-58M12 | RP11-644F13 |
| clone set: | RP11-813D1 | RP11-432N19 | RP11-58M12 | RP11-264K18 |
| clone set: | RP11-813D1 | RP11-432N19 | RP11-58M12 | RP11-264K18 |
| clone set: | RP11-813D1 | RP11-432N19 | RP11-58M12 | RP11-264K18 |
| clone set: | RP11-813D1 | RP11-432N19 | RP11-384D8 | RP11-533M23 |
| clone set: | RP11-813D1 | RP11-432N19 | RP11-384D8 | RP11-533M23 |
| clone set: | RP11-813D1 | RP11-432N19 | RP11-384D8 | RP11-533M23 |
| clone set: | RP11-813D1 | RP11-432N19 | RP11-384D8 | RP11-644F13 |
| clone set: | RP11-813D1 | RP11-432N19 | RP11-384D8 | RP11-644F13 |
| clone set: | RP11-813D1 | RP11-432N19 | RP11-384D8 | RP11-644F13 |
| clone set: | RP11-813D1 | RP11-432N19 | RP11-384D8 | RP11-264K18 |
| clone set: | RP11-813D1 | RP11-432N19 | RP11-384D8 | RP11-264K18 |
| clone set: | RP11-813D1 | RP11-432N19 | RP11-384D8 | RP11-264K18 |
| clone set: | RP11-813D1 | RP11-430B3 | RP11-98E9 | RP11-533M23 |
| clone set: | RP11-813D1 | RP11-430B3 | RP11-98E9 | RP11-533M23 |
| clone set: | RP11-813D1 | RP11-430B3 | RP11-98E9 | RP11-533M23 |
| clone set: | RP11-813D1 | RP11-430B3 | RP11-98E9 | RP11-644F13 |
| clone set: | RP11-813D1 | RP11-430B3 | RP11-98E9 | RP11-644F13 |
| clone set: | RP11-813D1 | RP11-430B3 | RP11-98E9 | RP11-644F13 |
| clone set: | RP11-813D1 | RP11-430B3 | RP11-98E9 | RP11-264K18 |
| clone set: | RP11-813D1 | RP11-430B3 | RP11-98E9 | RP11-264K18 |
| clone set: | RP11-813D1 | RP11-430B3 | RP11-98E9 | RP11-264K18 |
| clone set: | RP11-813D1 | RP11-430B3 | RP11-58M12 | RP11-533M23 |
| clone set: | RP11-813D1 | RP11-430B3 | RP11-58M12 | RP11-533M23 |
| clone set: | RP11-813D1 | RP11-430B3 | RP11-58M12 | RP11-533M23 |
| clone set: | RP11-813D1 | RP11-430B3 | RP11-58M12 | RP11-644F13 |
| clone set: | RP11-813D1 | RP11-430B3 | RP11-58M12 | RP11-644F13 |
| clone set: | RP11-813D1 | RP11-430B3 | RP11-58M12 | RP11-644F13 |
| clone set: | RP11-813D1 | RP11-430B3 | RP11-58M12 | RP11-264K18 |
| clone set: | RP11-813D1 | RP11-430B3 | RP11-58M12 | RP11-264K18 |
| clone set: | RP11-813D1 | RP11-430B3 | RP11-58M12 | RP11-264K18 |
| clone set: | RP11-813D1 | RP11-430B3 | RP11-384D8 | RP11-533M23 |
| clone set: | RP11-813D1 | RP11-430B3 | RP11-384D8 | RP11-533M23 |
| clone set: | RP11-813D1 | RP11-430B3 | RP11-384D8 | RP11-533M23 |
| clone set: | RP11-813D1 | RP11-430B3 | RP11-384D8 | RP11-644F13 |
| clone set: | RP11-813D1 | RP11-430B3 | RP11-384D8 | RP11-644F13 |
| clone set: | RP11-813D1 | RP11-430B3 | RP11-384D8 | RP11-644F13 |
| clone set: | RP11-813D1 | RP11-430B3 | RP11-384D8 | RP11-264K18 |
| clone set: | RP11-813D1 | RP11-430B3 | RP11-384D8 | RP11-264K18 |
| clone set: | RP11-813D1 | RP11-430B3 | RP11-384D8 | RP11-264K18 |
| clone set: | RP11-719G24 | RP11-813D1 | RP11-98E9 | RP11-533M23 |
| clone set: | RP11-719G24 | RP11-813D1 | RP11-98E9 | RP11-533M23 |
| clone set: | RP11-719G24 | RP11-813D1 | RP11-98E9 | RP11-644F13 |
| clone set: | RP11-719G24 | RP11-813D1 | RP11-98E9 | RP11-644F13 |
| clone set: | RP11-719G24 | RP11-813D1 | RP11-98E9 | RP11-264K18 |
| clone set: | RP11-719G24 | RP11-813D1 | RP11-98E9 | RP11-264K18 |
| clone set: | RP11-719G24 | RP11-813D1 | RP11-58M12 | RP11-533M23 |
| clone set: | RP11-719G24 | RP11-813D1 | RP11-58M12 | RP11-533M23 |
| clone set: | RP11-719G24 | RP11-813D1 | RP11-58M12 | RP11-644F13 |
| clone set: | RP11-719G24 | RP11-813D1 | RP11-58M12 | RP11-644F13 |
| clone set: | RP11-719G24 | RP11-813D1 | RP11-58M12 | RP11-264K18 |
| clone set: | RP11-719G24 | RP11-813D1 | RP11-58M12 | RP11-264K18 |
| clone set: | RP11-719G24 | RP11-813D1 | RP11-384D8 | RP11-533M23 |
| clone set: | RP11-719G24 | RP11-813D1 | RP11-384D8 | RP11-533M23 |
| clone set: | RP11-719G24 | RP11-813D1 | RP11-384D8 | RP11-644F13 |
| clone set: | RP11-719G24 | RP11-813D1 | RP11-384D8 | RP11-644F13 |
| clone set: | RP11-719G24 | RP11-813D1 | RP11-384D8 | RP11-264K18 |
| clone set: | RP11-719G24 | RP11-813D1 | RP11-384D8 | RP11-264K18 |
| clone set: | RP11-719G24 | RP11-432N19 | RP11-98E9 | RP11-533M23 |
| clone set: | RP11-719G24 | RP11-432N19 | RP11-98E9 | RP11-533M23 |
| clone set: | RP11-719G24 | RP11-432N19 | RP11-98E9 | RP11-644F13 |
| clone set: | RP11-719G24 | RP11-432N19 | RP11-98E9 | RP11-644F13 |
| clone set: | RP11-719G24 | RP11-432N19 | RP11-98E9 | RP11-264K18 |
| clone set: | RP11-719G24 | RP11-432N19 | RP11-98E9 | RP11-264K18 |
| clone set: | RP11-719G24 | RP11-432N19 | RP11-58M12 | RP11-533M23 |
| clone set: | RP11-719G24 | RP11-432N19 | RP11-58M12 | RP11-533M23 |
| clone set: | RP11-719G24 | RP11-432N19 | RP11-58M12 | RP11-644F13 |
| clone set: | RP11-719G24 | RP11-432N19 | RP11-58M12 | RP11-644F13 |
| clone set: | RP11-719G24 | RP11-432N19 | RP11-58M12 | RP11-264K18 |
| clone set: | RP11-719G24 | RP11-432N19 | RP11-58M12 | RP11-264K18 |
| clone set: | RP11-719G24 | RP11-432N19 | RP11-384D8 | RP11-533M23 |
| clone set: | RP11-719G24 | RP11-432N19 | RP11-384D8 | RP11-533M23 |
| clone set: | RP11-719G24 | RP11-432N19 | RP11-384D8 | RP11-644F13 |
| clone set: | RP11-719G24 | RP11-432N19 | RP11-384D8 | RP11-644F13 |
| clone set: | RP11-719G24 | RP11-432N19 | RP11-384D8 | RP11-264K18 |
| clone set: | RP11-719G24 | RP11-432N19 | RP11-384D8 | RP11-264K18 |
| clone set: | RP11-719G24 | RP11-430B3 | RP11-98E9 | RP11-533M23 |
| clone set: | RP11-719G24 | RP11-430B3 | RP11-98E9 | RP11-533M23 |
| clone set: | RP11-719G24 | RP11-430B3 | RP11-98E9 | RP11-644F13 |
| clone set: | RP11-719G24 | RP11-430B3 | RP11-98E9 | RP11-644F13 |
| clone set: | RP11-719G24 | RP11-430B3 | RP11-98E9 | RP11-264K18 |
| clone set: | RP11-719G24 | RP11-430B3 | RP11-98E9 | RP11-264K18 |
| clone set: | RP11-719G24 | RP11-430B3 | RP11-58M12 | RP11-533M23 |
| clone set: | RP11-719G24 | RP11-430B3 | RP11-58M12 | RP11-533M23 |
| clone set: | RP11-719G24 | RP11-430B3 | RP11-58M12 | RP11-644F13 |
| clone set: | RP11-719G24 | RP11-430B3 | RP11-58M12 | RP11-644F13 |
| clone set: | RP11-719G24 | RP11-430B3 | RP11-58M12 | RP11-264K18 |
| clone set: | RP11-719G24 | RP11-430B3 | RP11-58M12 | RP11-264K18 |
| clone set: | RP11-719G24 | RP11-430B3 | RP11-384D8 | RP11-533M23 |
| clone set: | RP11-719G24 | RP11-430B3 | RP11-384D8 | RP11-533M23 |
| clone set: | RP11-719G24 | RP11-430B3 | RP11-384D8 | RP11-644F13 |
| clone set: | RP11-719G24 | RP11-430B3 | RP11-384D8 | RP11-644F13 |
| clone set: | RP11-719G24 | RP11-430B3 | RP11-384D8 | RP11-264K18 |
| clone set: | RP11-719G24 | RP11-430B3 | RP11-384D8 | RP11-264K18 |
| clone set: | RP11-1093B19 | RP11-813D1 | RP11-98E9 | RP11-533M23 |
| clone set: | RP11-1093B19 | RP11-813D1 | RP11-98E9 | RP11-533M23 |
| clone set: | RP11-1093B19 | RP11-813D1 | RP11-98E9 | RP11-644F13 |
| clone set: | RP11-1093B19 | RP11-813D1 | RP11-98E9 | RP11-644F13 |
| clone set: | RP11-1093B19 | RP11-813D1 | RP11-98E9 | RP11-264K18 |
| clone set: | RP11-1093B19 | RP11-813D1 | RP11-98E9 | RP11-264K18 |
| clone set: | RP11-1093B19 | RP11-813D1 | RP11-58M12 | RP11-533M23 |
| clone set: | RP11-1093B19 | RP11-813D1 | RP11-58M12 | RP11-533M23 |
| clone set: | RP11-1093B19 | RP11-813D1 | RP11-58M12 | RP11-644F13 |
| clone set: | RP11-1093B19 | RP11-813D1 | RP11-58M12 | RP11-644F13 |
| clone set: | RP11-1093B19 | RP11-813D1 | RP11-58M12 | RP11-264K18 |
| clone set: | RP11-1093B19 | RP11-813D1 | RP11-58M12 | RP11-264K18 |
| clone set: | RP11-1093B19 | RP11-813D1 | RP11-384D8 | RP11-533M23 |
| clone set: | RP11-1093B19 | RP11-813D1 | RP11-384D8 | RP11-533M23 |
| clone set: | RP11-1093B19 | RP11-813D1 | RP11-384D8 | RP11-644F13 |
| clone set: | RP11-1093B19 | RP11-813D1 | RP11-384D8 | RP11-644F13 |
| clone set: | RP11-1093B19 | RP11-813D1 | RP11-384D8 | RP11-264K18 |
| clone set: | RP11-1093B19 | RP11-813D1 | RP11-384D8 | RP11-264K18 |
| clone set: | RP11-1093B19 | RP11-432N19 | RP11-98E9 | RP11-533M23 |
| clone set: | RP11-1093B19 | RP11-432N19 | RP11-98E9 | RP11-533M23 |
| clone set: | RP11-1093B19 | RP11-432N19 | RP11-98E9 | RP11-644F13 |
| clone set: | RP11-1093B19 | RP11-432N19 | RP11-98E9 | RP11-644F13 |
| clone set: | RP11-1093B19 | RP11-432N19 | RP11-98E9 | RP11-264K18 |
| clone set: | RP11-1093B19 | RP11-432N19 | RP11-98E9 | RP11-264K18 |
| clone set: | RP11-1093B19 | RP11-432N19 | RP11-58M12 | RP11-533M23 |
| clone set: | RP11-1093B19 | RP11-432N19 | RP11-58M12 | RP11-533M23 |
| clone set: | RP11-1093B19 | RP11-432N19 | RP11-58M12 | RP11-644F13 |
| clone set: | RP11-1093B19 | RP11-432N19 | RP11-58M12 | RP11-644F13 |
| clone set: | RP11-1093B19 | RP11-432N19 | RP11-58M12 | RP11-264K18 |
| clone set: | RP11-1093B19 | RP11-432N19 | RP11-58M12 | RP11-264K18 |
| clone set: | RP11-1093B19 | RP11-432N19 | RP11-384D8 | RP11-533M23 |
| clone set: | RP11-1093B19 | RP11-432N19 | RP11-384D8 | RP11-533M23 |
| clone set: | RP11-1093B19 | RP11-432N19 | RP11-384D8 | RP11-644F13 |
| clone set: | RP11-1093B19 | RP11-432N19 | RP11-384D8 | RP11-644F13 |
| clone set: | RP11-1093B19 | RP11-432N19 | RP11-384D8 | RP11-264K18 |
| clone set: | RP11-1093B19 | RP11-432N19 | RP11-384D8 | RP11-264K18 |
| clone set: | RP11-1093B19 | RP11-430B3 | RP11-98E9 | RP11-533M23 |
| clone set: | RP11-1093B19 | RP11-430B3 | RP11-98E9 | RP11-533M23 |
| clone set: | RP11-1093B19 | RP11-430B3 | RP11-98E9 | RP11-644F13 |
| clone set: | RP11-1093B19 | RP11-430B3 | RP11-98E9 | RP11-644F13 |
| clone set: | RP11-1093B19 | RP11-430B3 | RP11-98E9 | RP11-264K18 |
| clone set: | RP11-1093B19 | RP11-430B3 | RP11-98E9 | RP11-264K18 |
| clone set: | RP11-1093B19 | RP11-430B3 | RP11-58M12 | RP11-533M23 |
| clone set: | RP11-1093B19 | RP11-430B3 | RP11-58M12 | RP11-533M23 |
| clone set: | RP11-1093B19 | RP11-430B3 | RP11-58M12 | RP11-644F13 |
| clone set: | RP11-1093B19 | RP11-430B3 | RP11-58M12 | RP11-644F13 |
| clone set: | RP11-1093B19 | RP11-430B3 | RP11-58M12 | RP11-264K18 |
| clone set: | RP11-1093B19 | RP11-430B3 | RP11-58M12 | RP11-264K18 |
| clone set: | RP11-1093B19 | RP11-430B3 | RP11-384D8 | RP11-533M23 |
| clone set: | RP11-1093B19 | RP11-430B3 | RP11-384D8 | RP11-533M23 |
| clone set: | RP11-1093B19 | RP11-430B3 | RP11-384D8 | RP11-644F13 |
| clone set: | RP11-1093B19 | RP11-430B3 | RP11-384D8 | RP11-644F13 |
| clone set: | RP11-1093B19 | RP11-430B3 | RP11-384D8 | RP11-264K18 |
| clone set: | RP11-1093B19 | RP11-430B3 | RP11-384D8 | RP11-264K18 |
| clone set: | RP11-813D1 | RP11-813D1 | RP11-98E9 | RP11-533M23 |
| clone set: | RP11-813D1 | RP11-813D1 | RP11-98E9 | RP11-533M23 |
| clone set: | RP11-813D1 | RP11-813D1 | RP11-98E9 | RP11-644F13 |
| clone set: | RP11-813D1 | RP11-813D1 | RP11-98E9 | RP11-644F13 |
| clone set: | RP11-813D1 | RP11-813D1 | RP11-98E9 | RP11-264K18 |
| clone set: | RP11-813D1 | RP11-813D1 | RP11-98E9 | RP11-264K18 |
| clone set: | RP11-813D1 | RP11-813D1 | RP11-58M12 | RP11-533M23 |
| clone set: | RP11-813D1 | RP11-813D1 | RP11-58M12 | RP11-533M23 |
| clone set: | RP11-813D1 | RP11-813D1 | RP11-58M12 | RP11-644F13 |
| clone set: | RP11-813D1 | RP11-813D1 | RP11-58M12 | RP11-644F13 |
| clone set: | RP11-813D1 | RP11-813D1 | RP11-58M12 | RP11-264K18 |
| clone set: | RP11-813D1 | RP11-813D1 | RP11-58M12 | RP11-264K18 |
| clone set: | RP11-813D1 | RP11-813D1 | RP11-384D8 | RP11-533M23 |
| clone set: | RP11-813D1 | RP11-813D1 | RP11-384D8 | RP11-533M23 |
| clone set: | RP11-813D1 | RP11-813D1 | RP11-384D8 | RP11-644F13 |
| clone set: | RP11-813D1 | RP11-813D1 | RP11-384D8 | RP11-644F13 |
| clone set: | RP11-813D1 | RP11-813D1 | RP11-384D8 | RP11-264K18 |
| clone set: | RP11-813D1 | RP11-813D1 | RP11-384D8 | RP11-264K18 |
| clone set: | RP11-813D1 | RP11-432N19 | RP11-98E9 | RP11-533M23 |
| clone set: | RP11-813D1 | RP11-432N19 | RP11-98E9 | RP11-533M23 |
| clone set: | RP11-813D1 | RP11-432N19 | RP11-98E9 | RP11-644F13 |
| clone set: | RP11-813D1 | RP11-432N19 | RP11-98E9 | RP11-644F13 |
| clone set: | RP11-813D1 | RP11-432N19 | RP11-98E9 | RP11-264K18 |
| clone set: | RP11-813D1 | RP11-432N19 | RP11-98E9 | RP11-264K18 |
| clone set: | RP11-813D1 | RP11-432N19 | RP11-58M12 | RP11-533M23 |
| clone set: | RP11-813D1 | RP11-432N19 | RP11-58M12 | RP11-533M23 |
| clone set: | RP11-813D1 | RP11-432N19 | RP11-58M12 | RP11-644F13 |
| clone set: | RP11-813D1 | RP11-432N19 | RP11-58M12 | RP11-644F13 |
| clone set: | RP11-813D1 | RP11-432N19 | RP11-58M12 | RP11-264K18 |
| clone set: | RP11-813D1 | RP11-432N19 | RP11-58M12 | RP11-264K18 |
| clone set: | RP11-813D1 | RP11-432N19 | RP11-384D8 | RP11-533M23 |
| clone set: | RP11-813D1 | RP11-432N19 | RP11-384D8 | RP11-533M23 |
| clone set: | RP11-813D1 | RP11-432N19 | RP11-384D8 | RP11-644F13 |
| clone set: | RP11-813D1 | RP11-432N19 | RP11-384D8 | RP11-644F13 |
| clone set: | RP11-813D1 | RP11-432N19 | RP11-384D8 | RP11-264K18 |
| clone set: | RP11-813D1 | RP11-432N19 | RP11-384D8 | RP11-264K18 |
| clone set: | RP11-813D1 | RP11-430B3 | RP11-98E9 | RP11-533M23 |
| clone set: | RP11-813D1 | RP11-430B3 | RP11-98E9 | RP11-533M23 |
| clone set: | RP11-813D1 | RP11-430B3 | RP11-98E9 | RP11-644F13 |
| clone set: | RP11-813D1 | RP11-430B3 | RP11-98E9 | RP11-644F13 |
| clone set: | RP11-813D1 | RP11-430B3 | RP11-98E9 | RP11-264K18 |
| clone set: | RP11-813D1 | RP11-430B3 | RP11-98E9 | RP11-264K18 |
| clone set: | RP11-813D1 | RP11-430B3 | RP11-58M12 | RP11-533M23 |
| clone set: | RP11-813D1 | RP11-430B3 | RP11-58M12 | RP11-533M23 |
| clone set: | RP11-813D1 | RP11-430B3 | RP11-58M12 | RP11-644F13 |
| clone set: | RP11-813D1 | RP11-430B3 | RP11-58M12 | RP11-644F13 |
| clone set: | RP11-813D1 | RP11-430B3 | RP11-58M12 | RP11-264K18 |
| clone set: | RP11-813D1 | RP11-430B3 | RP11-58M12 | RP11-264K18 |
| clone set: | RP11-813D1 | RP11-430B3 | RP11-384D8 | RP11-533M23 |
| clone set: | RP11-813D1 | RP11-430B3 | RP11-384D8 | RP11-533M23 |
| clone set: | RP11-813D1 | RP11-430B3 | RP11-384D8 | RP11-644F13 |
| clone set: | RP11-813D1 | RP11-430B3 | RP11-384D8 | RP11-644F13 |
| clone set: | RP11-813D1 | RP11-430B3 | RP11-384D8 | RP11-264K18 |
| clone set: | RP11-813D1 | RP11-430B3 | RP11-384D8 | RP11-264K18 |

Depletion of CHD1 also affects DNA repair and sensitizes cells to PARP inhibitors, indicating that this class of drugs are useful targets for CHD1 mutations. (Kari et al. (EMBO Rep. 2016 November; 17(11):1609-1623. Epub 2016 Sep. 5). In addition to this, for prostate cancer, CHD1 deletion predicts a worse outcome, and thus early detection should be accompanied by aggressive treatment, such as radiation treatment, chemotherapy, and/or surgery. Treatments include dasatinib, abiraterone, enzalutamide, radiotherapy, surgery, androgen suppression, leuprorelin, zoledronic acid, zibotentan, ODM-201, orteronel, radium-223, ipilimumab, docetaxel, sunitinib, goserelin, bicalutamide, dutasteride, ketoconazole, tadalafil, atrasentan, denosumab, aflibercept, venlafaxine, docetaxel, androgen deprivation therapy (ADT) with luteinizing hormone-releasing hormone (LHRH) agonists, checkpoint inhibitors, taxol, cisplatin, and/or prednisone. See Foucher et al., Health Qual Life Outcomes. 2018; 16: 40; Meani et al., Ther Adv Urol. 2017 Nov. 23;10(2):51-63.

The detection of CHD1 can be used in combination with LSAMP. LSAMP deletions are described Ser. No. 15/862,343 (published as 2018/0135137 A1), which is incorporated by reference.

A useful probe can be made by selecting DNA, such as from a BAC clone, where two or more of the DNAs overlap with each other in such a way that the completed probe contains a higher representation of the overlapped region than regions which show no overlap. For instance, a probe can be designed utilizing overlapping middle regions of the CHD1 gene and non-overlapping 3′ and 5′ regions. The genomic clones listed in FIG. 3 are useful to produce deletion probes of CHD1.

### Chromosome Counting Probes

Embodiments also includes chromosome counting probes. Such probes can be used to count the chromosomes, e.g., in metaphase spread, and/or to detect specific chromosomes. For example, probes to chromosome centromere regions can be prepared from centromeric DNA using specific primers. The control probes show in FIG. 4 can be used as counting probes for chromosome 5. FIG. 4 is a list of genomic clones for the design of control probes matching the D5S23 and D5S721 genomic region. A control probe can comprise from 1 to about 10 sequences.

### EXAMPLES

### Example 1

Generate fluorescence labeled DNA probes by Nick Translation. DNA was extracted from identified BAC clones. Labeling was performed in two steps: nick translation introducing aminoallyl-dUTP and chemical coupling of an amine-reactive dye. Specifically, DNase I was used to create single-strand breaks, then DNA polymerase I was used to elongate the 3′ ends of these “nicks”, replacing existing nucleotides with new aminoallyl-dUTP. The fluorescent labeling of the probe was completed by chemical coupling of the dye. Alexa Fluor succinimidyl ester dyes react with the amines of the amino-allyl-dUTP modified DNA, thereby forming fluorescently labeled probes. Standard Ethanol precipitation method was used to isolate the fluorescently labeled probe. The probe pellet was suspended in deionized formamide/dextran sulphate.

DNA FISH Protocol. Cell slides were pretreated in pepsin solution before undergoing fixation in formaldehyde, followed by serial ethanol dehydration. The slides were denatured in formamide/saline sodium citrate (SSC) solution, followed by ice cold dehydrating ethanol series. Probes were denatured at 80° C. followed by a pre-annealing step. Pre-annealed probes were added to the denatured slides. The slides were then cover-slipped and sealed for overnight hybridization in a humidified chamber. After hybridization, slides were washed and dehydrated. At last, the slides were counterstained with anti-fade solution and mounted with coverslip for observation.

### Example 2: Study of CHD1 Deletions in Prostate Cancers of African American

### Experimental

### Cohort Selection and Tissue Microarray (TMA) Generation

The aggregate cohort was composed of 2 independently selected cohort samples from Bio-specimen bank of Center for Prostate Disease Research and the Joint Pathology Center. Whole mount prostates were collected from 1996 to 2008 with minimal follow-up time of 10 years. The first cohort of 42 AA and 59 EA cases was described before^{7,38}. Similarly, the second cohort of 50 AA and 50 EA cases was selected based on the tissue availability (>1.0 cm tumor tissue) and tissue differentiation status (⅓ well differentiated, ⅓ moderately differentiated and ⅓ poorly differentiated). All the selected cases had the signed patient consent forms for tissue research applications. Patients who have donated tissue for this study also contributed to the long term follow-up data (mean 14.5 years). This study was reviewed and approved by institutional review board (IRB) of WRNMMC and Uniformed Services University of the Health Sciences, Bethesda, Md. TMA block was assigned as 10 cases each slide and each case with 2 benign tissue cores, 2 Prostatic intraepithelial neoplasia (PIN) cores if available and 4-10 tumor cores covering the index and non-index tumors from formalin fixed paraffin embedded (FFPE) whole mount blocks. All the blocks were sectioned into 8 μM tissue slides for FISH staining.

Fluorescence in situ hybridization (FISH) assay: A gene-specific FISH probe for CHD1 was generated by selecting a combination of bacterial artificial chromosome (BAC) clones (Thermo Fisher Scientific, Waltham, Mass.) within the region of observed deletions near 5q15-q21.1, resulting in a probe matching ca. 430 kbp covering the CHD1 gene as well as some upstream and downstream adjacent genomic sequences including the complete repulsive guidance molecule B (RGMB) gene. Due to the high degree of homology of chromosome 5-specific alpha satellite centromeric DNA to the centromere repeat sequences on other chromosomes, and the resulting potential for cross-hybridization to other centromere sequences, particularly on human chromosomes 1 and 19, a control probe matching a stable genomic region on the short arm of chromosome 5—instead of a centromere 5 probe—was used for chromosome 5 counting. The FISH assay of CHD1 was performed on TMA as previously described. The green signal was from probe detecting control chromosome 5 short arm, and the red signal was from probe detecting CHD1 gene copy. The FISH stained TMA slides were scanned with Leica Aperio VERSA digital pathology scanner for further evaluation. The criteria for CHD1 deletion was that in over 50% of counted cancer cells (with at least 2 copies of chromosome 5 short arm detected in one tumor cell) more than one copy of CHD1 gene had to be undetected. Examining tumor cores, deletions were called when more than 75% of evaluable tumor cells showed loss of allele. Focal deletions were called when more than 25% of evaluable tumor cells showed loss of allele or when more than 50% evaluable tumor cells in each gland of a cluster of two or three tumor glands showed loss of allele. Benign prostatic glands and stroma served as built-in control.

The sub-clonality of CHD1 deletion was presented with a heatmap showing CHD1 deletion status in all the given tumors sampled from whole-mount sections of each patient. The color designations were denoted as: red color (full deletion) meaning all the tumor cores carrying CHD1 deletion within a given tumor, yellow color (sub-clonal deletion) meaning only partial tumor cores carrying CHD1 deletion within a given tumor and green color (no deletion) meaning no tumor core carry CHD1 deletion.

Statistics Analysis: The correlations of CHD1 deletion and clinic-pathological features, including pathological stages, Gleason score sums, Grade groups, margin status, and therapy status were calculated using an unpaired t-test or chi-square test. Gleason Grade Groups were derived from the Gleason patterns for cohort from Grade group 1 to Grade group 5. Due to the small sample sizes within each Grade group, Grade group 1 through Grade group 3 were categorized as one level as well as Grade group 4 through Grade group 5. A BCR was defined as either two successive post-RP PSAs of ≥0.2 ng/mL or the initiation of salvage therapy after a rising PSA of ≥0.1 ng/mL. A metastatic event was defined by a review of each patient's radiographic scan history with a positive metastatic event defined as the date of a positive CT scan, bone scan, or MRI in their record. The associations of CHD1 deletion and clinical outcomes with time to event outcomes, including BCR and metastasis, were analyzed by a Kaplan-Meier survival curves and tested using a log-rank test. Multivariable Cox proportional hazards models were used to estimated hazard ratios (HR) and 95% confidence intervals (Cis) to adjust for age at diagnosis, PSA at diagnosis, race, pathological tumor stage, grade group, and surgical margins. The proportional hazards assumption was checked by plotting the log-log survival curves. A P-value <0.05 was considered statistically significant. Analyses were performed in R version 4.0.2.

### Immunohistochemistry for ERG

ERG immunohistochemistry was performed as previously described³⁹. Briefly, four m TMA sections were dehydrated and blocked in 0.6% hydrogen peroxide in methanol for 20 min and were processed for antigen retrieval in EDTA (pH 9.0) for 30 min in a microwave followed by 30 min of cooling in EDTA buffer. Sections were then blocked in 1% horse serum for 40 min and were incubated with the ERG-MAb mouse monoclonal antibody developed at CPDR (9FY, Biocare Medical Inc.) at a dilution of 1:1280 for 60 min at room temperature. Sections were incubated with the biotinylated horse anti-mouse antibody at a dilution of 1:200 (Vector Laboratories) for 30 min followed by treatment with the ABC Kit (Vector Laboratories) for 30 min. The color was developed by VIP (Vector Laboratories) treatment for 5 min, and the sections were counter stained by hematoxylin. ERG expression was reported as positive or negative. ERG protein expression was correlated with clinico-pathologic features.

### Prostate Cancer Patients and Specimens in the in Silico Study Cohorts

### Evaluation of the Self-Declared Ancestries

Since the available ancestry data were based on the self-assessment of the patients, and it was a crucial part of this study to identify the samples accurately, the genotypes of 3000 SNPs that are specific to one of the greater Caucasian, African and Asian ancestries were investigated in each of the germline samples⁴⁰. The data was collected into a single genotype matrix, the first two principal components of which was used to train a non-naïve Bayes classifier to differentiate between the three ancestries.

### Identification of Local Subclonal Loss of CHD1 in Prostate Adenocarcinoma:

The paired germline and tumor binary alignment (bam) files were analyzed using bedtools genomcecov (v2.28.0)⁴¹, and their mean sequencing depths were determined. The coverage above and within the direct vicinity of CHD1 (chr5:98,853,485-98,930,272 in grch38 and chr5:98,190,408-98,262,740 in grch37) was collected in 50 bp wide bins into d-dimensional vectors (d_grch37=1447, d_grch38=1536) using an in-house tool and samtools (v1.6)⁴², and were normalized using their corresponding mean sequencing depths. The linear relationship between the paired germline-tumor coverages were determined in the following form:

cₙ=α+β₀cₜ,

where cₙ is the normalized coverage of the germline sample and cₜ is the normalized coverage of its corresponding tumor pair. The intercept (α) was used to ensure that the data was free of outliers, and the slope (β₀) was used as a raw measure of the observable loss in the tumor. Similar slopes were calculated for 14 housekeeping genes in each of the sample-pairs, which were used to assess the significance of the loss.

The cellularity (c) of the tumors were estimated using sequenza⁴³ after the rigorous selection of the most reliable cellularity-ploidy pair offered by the tool as alternative solutions. In order to account for the uncertainty of the reported cellularity values, a beta distribution was fitted on the grid-approximated marginal posterior densities of c. These were used to simulate random variables to determine the proportion of the approximate loss of CHD1 in the tumors, by the following formula:

$${β}_{t} = \frac{β - 1 + c}{c}$$

Here, β˜Normal(β₀, σ), where a is the standard error of β₀, c˜Beta(s₁, s₂), where sₜ and s₂ are the fitted shape-parameters of the cellularity, and βₜ is the cellularity-adjusted slopes of the curve. The approximate level of loss in CHD1 is distributed as 1−βₜ.

### Local Subclonal LOH-Calling:

The SNP variant allele frequencies (VAF) in the close vicinity of CHD1 in the tumor were collected with GATK HaplotypeCaller (v4.1.0)⁴⁴. The coverage and VAF data were carefully analyzed in order to ensure that regions that have suffered the most serious loss (e.g., if only a part of the gene were lost, the unaffected region was excluded from the analysis) were focused on. By using the tumor cellularity (c) and the estimated level of loss in the tumor WO, whether a heterozygous or a homozygous subclonal deletion is more likely to result in the observed frequency pattern was assessed.

### Cell Culture Models.

PC-3 prostate cell line was purchased from ATCC® and grown in RPMI 1640 (Gibco) supplemented with 10% FBS (Gibco). RPE1-CHD1 knock out cells were provided by Alan D. D'Andrea's laboratory. Cells were incubated at 37° C. in 5% CO2, and regularly tested for Mycoplasma spp. contamination.

### Stable CRISPR-Cas9 Expressing Isogenic PC-3 Cell Line Generation.

Full length SpCas9 ORF was introduced in PC-3 cell population by Lentiviral transduction using lentiCas9-Blast (Addgene #52962) construction. After antibiotics (blasticidin) selection, survival populations were single cell cloned, isogenic cell lines were generated and tested for Cas9 activity by cleavage assay.

### Gene Knock-Out Induction.

CHD1 was targeted in stable CRISPR-Cas9 expressor isogenic PC-3 cell line using guide RNA CHD1_ex2_g1 (caccgCTGACTGCCTGATTCAGATC), resulting in PC-3 chd1 ko 1, and chd1 ko 2 homozygous loss cell lines.

### Transfection.

Cells were transiently transfected by Nucleofector® 4D device by using supplemented, Nucleofector® SF solution and 20 μl Nucleocuvette® strips following the manufacturer's instructions. Following transfection, cells were resuspended in 100 μl culturing media and plated in 1.5 ml pre-warmed culturing media in a 24 well tissue culture plate. Cells were subjected to further assays 72 h post transfection.

### In Vitro T7 Endonuclease I (T7E1) Assay.

Templates used for T7E1 were amplified by PCR using CGTCAACGATGTCACTAGGC forward and ATGATTTGGGGCTTTCTGCT reverse oligos generating a 946 bp amplicon. 500 ng PCR products were denatured and reannealed in 1× NEBuffer 2.1 (New England Biolabs) using the following protocol: 95° C., 5 min; 95-85° C. at −2° C./sec; 85-25° C. at −0.1° C./sec; hold at 4° C. Hybridized PCR products were then treated with 10 U of T7E1 enzyme (New England Biolabs) for 30 min in a reaction volume of 30 μl. Reactions were stopped by adding 2 μl 0.5 M EDTA, fragments were visualized by agarose gel electrophoresis.

### Immunoblot Analysis.

Freshly harvested cells were lysed in RIPA buffer. Protein concentrations were determined by Pierce BCA™ Protein Assay Kit (Pierce). Proteins were separated via Mini Protean TGX stain free gel 4-15% (BioRad) and transferred to polyvinilydene difluoride membrane by using iBlot 2 PVDF Regular Stacks (Invitrogene) and iBlot system transfer system (LifeTechnologies).

Membranes were blocked in 5% BSA solution (Sigma). Primary antibodies were diluted following the manufacturer's instructions: anti-Vinculin antibody (Cell Signaling) (1:1000) and antiCHD1 (Novus Biologicals) (1:2000).

Signals were developed by using Clarity Western ECL Substrate (BioRad) and Image Quant LAS4000 System (GEHealthCare).

### Sample Preparation for Whole Genome Sequencing (WGS).

RPE1 DNA was extracted from wt and chd1 knock out isogenic cell lines at low passage number of the cells. Following 45 passages, CHD1 knock out isogenic cell line was single cell cloned, and two colonies were propagated for DNA isolation.

DNA was extracted by using QIAamp DNA Mini Kit (QIAGENE). Whole Genome Sequencing of the DNA samples was carried out at Novogene and BGI service companies.

### Viability Cell Proliferation Assays.

Exponentially growing PC-3 cell lines wt, chd1 ko1, chd1 ko2 were seeded in 96-well plates (1000 cells/well) and incubated for 36 hrs to facilitate cell attachment. Identical cell numbers of seeded parallel isogenic lines were verified by the Celigo Imaging Cytometer after attachment.

Cells were exposed to Talazoparib (Selleckchem), Olaparib (MedChemExpress) and Bleomycin sulfate (Fisher Scientific) for 24 hrs, then kept in drug-free fresh media for 5 days until cell growth was determined by the addition of PrestoBlue (Invitrogen) and incubated for 2.5 hrs. Cell viability was determined by using the BioTek plate reader system. Fluorescence was recorded at 560 nm/590 nm, and values were calculated based on the fluorescence intensity. IC50 values were determined by using the AAT Bioquest IC50 calculator tool. P-values were calculated using student's t-test. P-values <0.05 were considered statistically significant.

### NGS Analysis of the RPE1 Whole Genomes Sequences

The reads of the four RPE1 WGS (1 parental and 3 CHD1 ko) were aligned to the grch37 reference genome using the bwa-mem⁴⁵ aligner. The resulting bam files were post-processed according to the GATK best-practices guidelines. Novel variants were called using Mutect2 (v4.1.0) by using the parental clone as “normal” and the CHD1 ko clones as “tumor” specimens⁴⁴. These vcfs were converted into tab-delimited files and further analyzed in R. Annotation was performed via Intervar⁴⁶.

### Results

Subclonal CHD1 Deletion is More Frequent in African American Prostate Cancers with Worse Clinical Outcome.

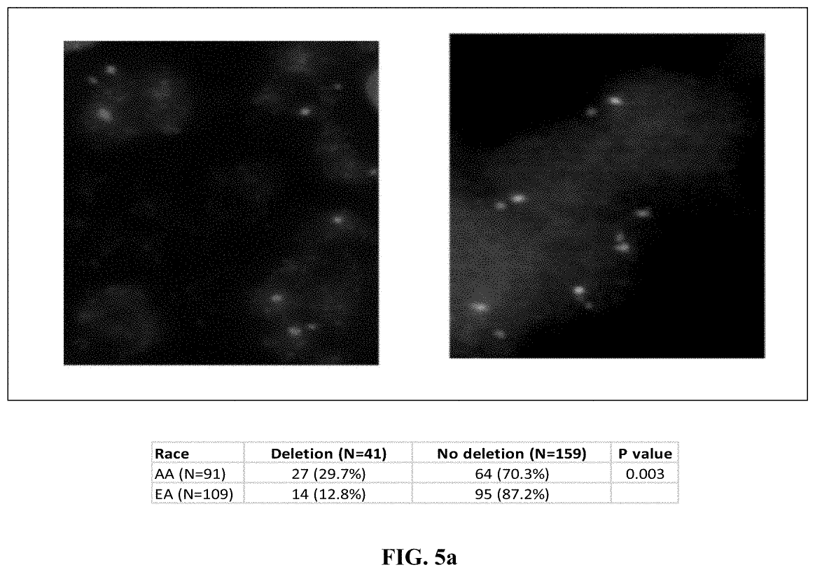

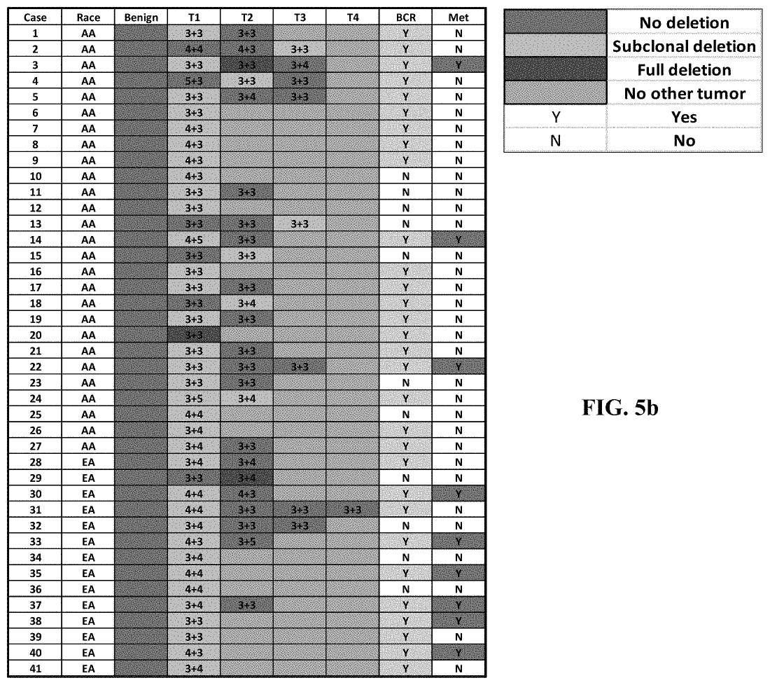

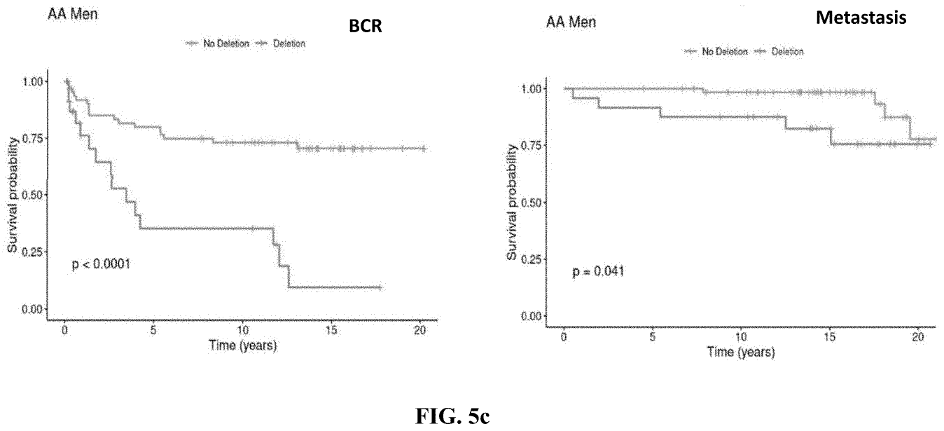

CHD1 is frequently subclonally deleted in prostate cancer¹⁸. The initial analysis on the SNP array data from TCGA comparing AA and EA PC cases suggested that the subclonal loss of CHD1 may be a more frequent event in AA men. To independently validate this observation, CHD1 copy number was assessed by FISH in tissue microarrays (TMAs) constructed from multiple tumor foci per prostatectomy specimen in a matched cohort of 91 AA and 109 EA patients from the equal-access military healthcare system (FIG. 5A). Key clinico-pathological features including diagnosis, age, diagnosis PSA levels, pathological stages, Gleason sums, Grade groups, margin status, adjuvant therapy, biochemical recurrence (BCR) and metastasis did not differ between AA and EA cases. Consistent with the long-term follow up (median: 16 years) of the cohort, it was observed a 40% biochemical recurrence (BCR) and 16% metastasis rate²⁰. For each clinical case up to four different cancerous areas were analyzed comprising 4-10 different tissue cores. xxx

It was detected subclonal CHD1 loss in 27 out of 91 AA cases (29.7%), and 14 out of 109 (11%) EA cases indicating that CHD1 deletion is about three times more frequent in prostate tumors of AA men. FISH data confirmed the subclonal nature of CHD1 deletion in prostate cancer cells (FIG. 5B which shows CHD1 deletion is a subclonal event in prostate cancer. Multiple tumor samples from 200 patients were assessed by FISH assay that identified 41 patients with CHD1 deletion (left panel). The heatmap depicts the sampled largest tumor 1 (T1), second largest tumor (T2), and so on. Numbers denote pathological Gleason grade for each tumor. In most cases CHD1 deletion was present in only a subset of tumor cores. As a control, FISH staining of PTEN and immunohistochemistry (IHC) staining of ERG were performed in a subset of the cohort (42 AA and 59 EA prostate cancer cases) confirming previously described differences^{4,5}.

Further analyses revealed a significant association between CHD1 deletion and pathologic stages and Gleason sum. Higher frequency of CHD1 deletion was detected in T3-4 pathological stage compared to T2 stage (P=0.043). Prostate cancer cases with higher Gleason sum scores (3+4, 4+3, 8-10) were seen more frequently in the CHD1 deletion group than in the non-deletion group (p<0.001). In contrast, lower Gleason sum score (3+3) was more often seen in non-deletion cases (P<0.001, table 1c). Notably, CHD1 deletion was strongly associated with rapid biochemical recurrence (FIG. 5C, which shows deletion of CHD1 is strongly associated with disease progression in AA prostate cancer patients (N=91) (P<0.0001)). The multivariate Cox model analysis showed that CHD1 deletion was an independent predictor of BCR in the entire cohort (P=0.0006) after adjusting for age at diagnosis, PSA at diagnosis, race, pathological tumor stage, grade group, and surgical margins. Moreover, a significant correlation between CHD1 deletion and metastasis was also detected in AA patients with Kaplan-Meier analysis (P=0.041) and multivariate logistic regression model (P=0.047). Following adjustment for age at diagnosis, PSA at diagnosis, race, pathological tumor stage, grade group, and surgical margins in the multivariate logistic regression model, CHD1 deletion was significantly associated with metastasis (P=0.047). Taken together, the data strongly support the association of CHD1 deletions with aggressive prostate cancer and worse clinical outcomes in AA PC.

### Estimating the Frequency of Subclonal CHD1 Loss in Next Generation Sequencing Data of AA and EA Prostate Cancer.

Previous publications characterizing the genome of AA prostate cancer cases^{10,21} did not report an increased frequency of CHD1 loss as it was observed in the FISH-based analysis presented above. Methods to detect copy number variations from WGS or WES data have at least two major limitations. First, subclonal copy number variations (sCNV) can be missed if they are present in fewer than 30%, of the cells¹⁹. Second, copy number loss can be underestimated with smaller deletions (e.g., <10 kb). Although various tools are available for inferring sCNVs from WES, WGS or SNP array data, such as TITAN¹⁹, THetA²², and Sclust²³, they are designed to work on the entire genome, and likely miss small (˜1-10 kb) CNVs during the data segmentation process. In order to maximize the accuracy of the analysis a gene focused analysis of the copy number loss in CHD1 was performed. Several factors were considered such as the change in the normalized coverage in the tumors relative to their normal pairs, the cellularity of the tumor genome, and the approximate proportion of tumor cells exhibiting the loss. It was also evaluated whether the deletion was heterozygous or homozygous using a statistical method designed for calling subclonal loss of heterozygosity (LOH) events within a confined genomic region.

Using this approach in a large cohort (N=530 cases; 59 AA WES, 18AA WGS, 408 EA WES and 45 EA WGS), it was observed that CHD1 is more frequently deleted in AA tumors (N=20; 26%) than in EA tumors (N=73 EA; 16%). Taken together, when next generation sequencing based copy number variations were analyzed with a more sensitive method, CHD1 loss was detected more frequently in the AA cases than in the EA cases (p=0.029, Fisher exact test), which is consistent with these observations with FISH method in the TMA cohort.

CHD1 Loss is Associated with Genomic Signatures Frequently Observed in BRCA2 Deficient Prostate Cancer.

CHD1 loss was shown to reduce HR competence in cell line model systems^{15,24}. Detecting and quantifying HR deficiency in tumor biopsies is currently best achieved by analyzing whole genome sequencing data for specific HR deficiency associated mutational signatures. Those include: 1) A single nucleotide variation based mutational signature (“COSMIC signatures 3²⁵ and SBS3²⁶); 2) a short insertions/deletions based mutational profile, often dominated by deletions with microhomology, a sign of alternative repair mechanisms joining double-strand breaks in the absence of HR, which is also captured by COSMIC indel signatures ID6 and ID8²⁶; 3) large scale rearrangements such as non-clustered tandem duplications in the size range of 1-100 kb (mainly associated with BRCA1 loss of function) or deletions in the range of 1 kb-1 Mb (mainly associated with BRCA2 loss of function)²⁷. All of these signatures can be efficiently induced by the inactivation of BRCA1, BRCA2 or several other key downstream HR genes²⁸.

HR deficiency is also assessed in the clinical setting by a large scale genomic aberration based signature, namely the HRD score²⁹, which is also approved as companion diagnostic for PARP inhibitor therapy. Recently a composite mutational signature, HRDetect³⁰, combining several of the mutational features listed above was evaluated as an alternative method to detect HR deficiency in prostate adenocarcinoma³¹. In order to further strengthen the link between CHD1 loss, HR deficiency and potentially increased PARP inhibitor sensitivity a detailed analysis on the mutational signature profiles of CHD1 deficient prostate cancer was performed.

Whole exome and whole genome sequencing data of several prostate adenocarcinoma cohorts containing samples both from AA (52 WES and 18 WGS cases) and EA (387 WES and 45 WGS cases) individuals were analyzed in order to determine whether CHD1 loss is associated with the HRD mutational signatures.

The cohorts were divided into three groups: 1) BRCA2 deficient cases that served as positive controls for HR deficiency, 2) CHD1 deleted cases without mutations in HR genes, and 3) cases without BRCA gene aberration or CHD1 deletion.

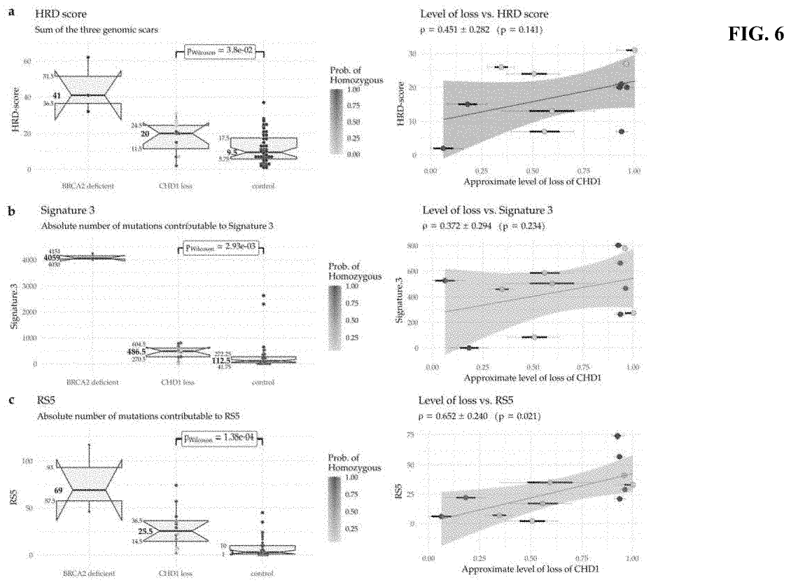

In the WGS cohorts CHD1 deficient cases showed increased HRD score relative to the control cases but lower than the BRCA2 deficient cases (FIG. 6a). It is important to emphasize, however, that the HRD score was positively correlated with the estimated fraction of the subclonal loss of CHD1 (FIG. 6b), suggesting that the signal of HRD score was “diluted” likely due to subclonality. The most characteristic HRD associated single nucleotide variation signature (signature 3) was significantly increased in the BRCA2 deficient cases and slightly increased in the CHD1 deficient cases (FIG. 6b).

The increase of the relative contribution of short indel signatures ID6 and ID8 to the total number of indels characteristic of loss of function on BRCA2 biallelic mutants was not observed in the CHD1 loss cases. This suggests that the alternative end-joining repair pathways do not dominate the repair of DSBs in those cases.

In the WGS cohort it was also determined the number of structural variants as previously defined²⁷. As expected, RS5 was significantly increased in the BRCA2 mutant cases since this signature (an increase in the number of non-clustered 1 kb-1 Mb deletions) was identified as a specific feature of such tumors. CHD1 deficient cases also displayed a significant increase in RS5 structural variations but the signal showed a strong subclonal dilution (FIG. 6c) suggesting that the number of RS5 aberrations may be similar in the BRCA2 and CHD1 deficient cases. Finally, the BRCA2 deficient cases showed high HRDetect scores. The HRDetect scores were also elevated relative to the controls but significantly lower than the previously published threshold for HR deficiency. However, since the HRDetect scores arise from a logistic regression, which involves the non-linear transformation of the weighted sum of its attributes, even slightly lower linear sums in the CHD1 loss cases compared to the BRCA2 mutant cases can result in substantially lower HRDetect scores.

WES prostate adenocarcinoma data were previously processed for the various HR deficiency associated mutational signatures³¹. When the CHD1 deficient cases were compared to the BRCA1/2 deficient and BRCA1/2 intact cases results were obtained that were consistent with the WGS based results outlined above.

### Deleting CHD1 in Cell Lines Induce Some Aspects of Homologous Recombination Deficiency-Associated Mutational Signatures

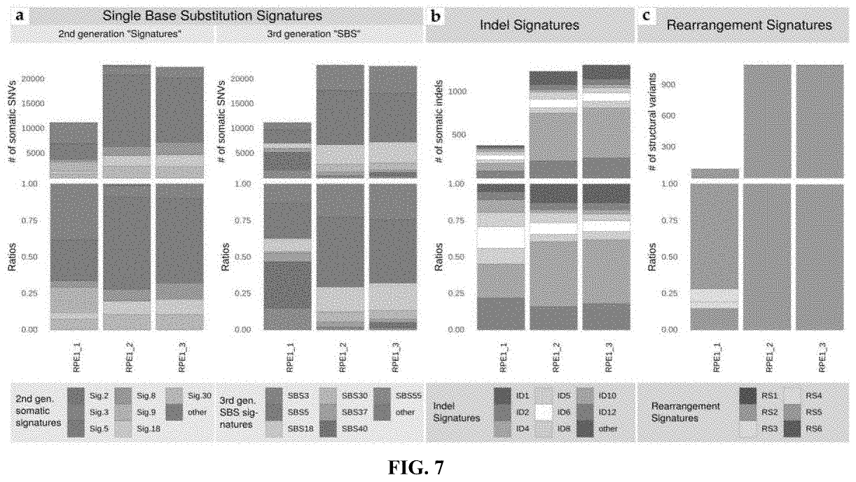

In order to investigate the functional impact of the biallelic loss of CHD1 several CRISPR-Cas9 edited cell lines were created. DNA repair pathway aberration induced mutational signatures can be detected in cell lines by whole genome sequencing^{28,32}. This analysis is more efficient if the starting cell line has a diploid genome, such as in the RPE1 (retinal pigment epithelium) cells in which CHD1 was previously deleted using CRISPR-Cas9 editing¹⁵. Single cell clones were grown from these cell lines for more than 45 generations to accumulate the genomic aberrations induced by CHD1 loss. Two of such late passage clones and an early passage clone were subjected to WGS analysis (FIG. 7xxxA). All of them retained the BRCA2 wild type background of their parental clone.

CHD1 elimination induced some increase in SBS3 (HR deficiency associated) but more significant increase in SBS5, SBS18 (FIG. 7B). Short indels flanked by microhomology (ID6 signature) constitute a robust sign of HR deficiency as a result of microhomology mediated-end joining (MMEJ) repair of DSBs in the absence of HR³³. CHD1 loss did not induce a significant increase of this mutational signature. Instead, the most significant increase was observed in ID10 (FIG. 7C). Large genomic rearrangement signatures showed a significant increase in RS5, the mutational signature strongly associated with BRCA2 loss, which was detected in prostate cancer cases with CHD1.

Taken together, CHD1 loss in cell line model systems mainly induced deletions of 1-10 kb, but only modestly induced the other types of mutational signatures that are associated with the loss of key members of the HR machinery.

### CHD1 Deficient Cell Lines Show Significantly Increased Sensitivity to Talazoparib and the Radiomimetic Agent Bleomycin.

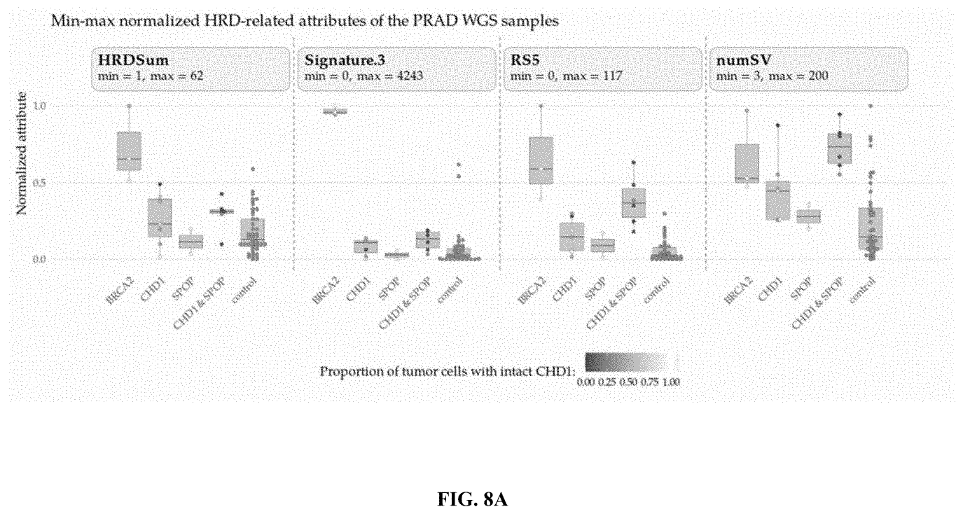

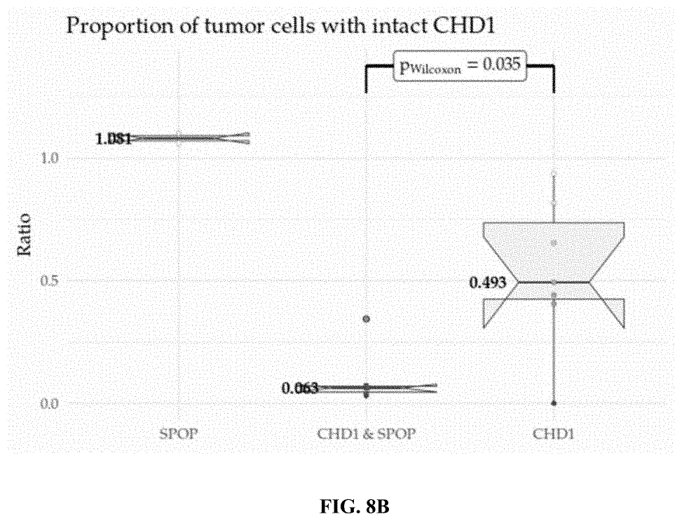

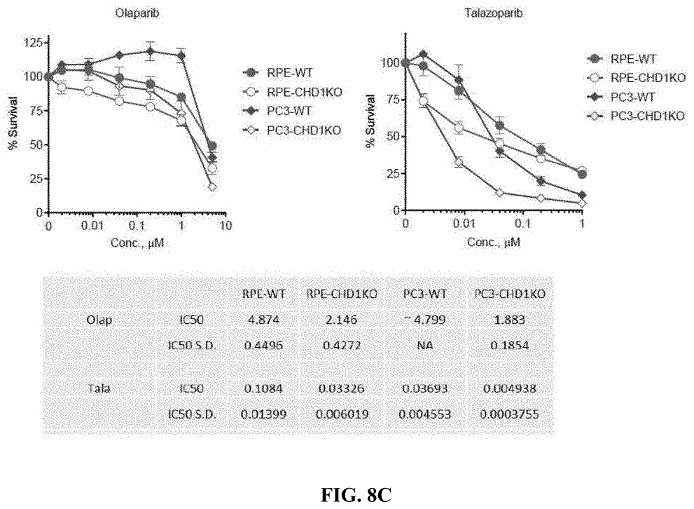

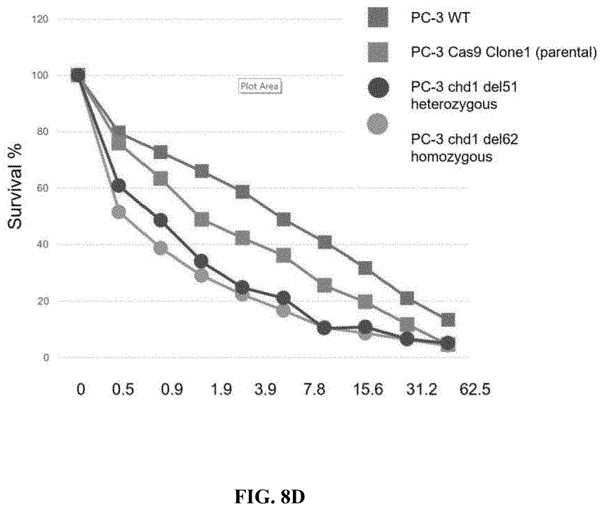

CHD1 deficient cancer cells have an increased sensitivity to the PARP inhibitor olaparib¹⁵. While this synthetic-lethal relationship is worth investigating, the olaparib sensitivity of CHD1-deficient is relatively mild. PARP inhibitors were initially thought to exert their therapeutic activity by inhibiting the enzymatic activity of PARP, but it was later revealed that trapped PARP on DNA may have a more significant contribution to cytotoxicity (reviewed in³⁴). Therefore, the efficacy of the strong PARP trapping agent talazoparib was tested in the prostate cancer cell line PC-3 with or without CRISPR-Cas9-mediated CHD1 deletion. CHD1 knock out clones were identified by immunoblotting (FIG. 8A). Consistent with previous reports, deleting CHD1 induced an approximately 5-fold increase in olaparib sensitivity (FIG. 8B). In contrast, the sensitivity to talazoparib increased by about 15-20-fold in the same CHD1 deficient PC-3 cells (FIG. 8C). These data suggest that trapped PARP may have a more toxic effect in cells with impaired CHD1 deficiency.

Consistent with the significant functional evidence linking CHD1 deletion and HR repair of DSBs, CHD1 deficient cells also showed increased sensitivity to irradiation¹⁵. Whether this increased sensitivity also applies to chemotherapy agents that induce DSBs, such as radiomimetic drugs, was investigated. As shown in FIG. 8D, CHD1 deficient cells show significantly increased (5-fold) sensitivity to bleomycin, the most frequently used radiomimetic therapeutic agent.

### The Impact of SPOP Mutations on the Clonality of CHD1 Deletions and HR Deficiency Associated Mutational Signatures.

SPOP mutations and CHD1 deletions show a strong tendency to co-exist in prostate cancer³⁵, and SPOP mutations have been shown to suppress key HR genes″. Therefore, it was investigated investigated whether the presence of SPOP mutation in a CHD1 deficient prostate cancer is associated with a further increase of HR deficiency associated mutational signatures. Cases with SPOP mutations or CHD1 deletions only were identified, cases with both SPOP mutations and CHD1 deletions and cases without either of those aberrations (FIG. 5A). Cases with both mutations showed significantly higher levels of signature SBS3, RS5 and the total number of large-scale structural rearrangements relative to cases with either mutation alone. It should be noted, however, that the proportion of cells with CHD1 deletions tended to be significantly higher in SPOP mutant cases than those with CHD1 deletions without SPOP mutations. Thus, considering the previously demonstrated impact of CHD1 subclonality on the intensity of HR deficiency associated mutational signatures, it is possible that the presence of SPOP will intensify HR deficiency associated mutational signatures by enhancing the proportion of CHD1 deficient cells in a tumor.

### Discussion

The presence of functionally relevant subclonal mutations in various solid tumor types is well documented^{36,37}. Deletions present only in a minority of tumor cells are difficult to detect unless more targeted analytical approaches are applied. Here one example of such detection bias is presented with significant functional relevance. A FISH based approach is used to detect CHD1 deletion in PC. Consistent with the previously described subclonal nature of CHD1 loss, it was found that while this gene is often deleted in prostate cancer, it is rarely deleted in every tumor cell. When the subclonal nature of CHD1 loss was taken into consideration a significant racial disparity emerged, with an approximately 3-fold increase in the frequency of CHD1 deletion in AA PC patients. This loss was also significantly associated with early biochemical recurrence. Since CHD1 loss is associated with a more malignant phenotype, the significantly higher frequency of CHD1 loss in AA PC may account for the diverging clinical course observed in PC between men of African and European Ancestry. It is possible that CHD1 loss is in fact more frequent in EA PC as well but with a lower focal density than in AA cases. This is certainly a limitation of this study, but with the sensitivity thresholds established the difference between AA and EA are significant.

Several studies pointed out a potentially intimate link between CHD1 loss an homologous recombination deficiency^{15,16,24}. Interestingly, CHD1 null cells showed only a modest (3-fold) increase in sensitivity to PARP inhibitor or platinum-based therapy^{15,16,24}. This suggested that CHD1 loss may not lead to the same level or the same completeness of HR deficiency as that detected upon loss of function of BRCA1 or BRCA2. The loss of function of those key HR genes usually leads to various DNA repair deficiencies such as stalled fork destabilization or reduced capacity of DSB repair. The presence of those DNA repair deficiencies can often be detected by different types of DNA aberration profiles, and they can be associated with an up to 1000-fold increase in PARP inhibitor sensitivity. The modest increase in PARP inhibitor sensitivity suggests that CHD1 loss may lead to some but not all DNA repair aberrations usually associated with loss of function of BRCA1/2. Indeed, CHD1 deficient tumors and cell line models displayed strong signals of the BRCA2 deficiency associated structural variation signature (SV5), but only modest or no increase of the single nucleotide variation or short indel based signatures. This suggests that CHD1 loss “mimics” some but not all of the consequences of BRCA2 deficiency. The precise mechanistic nature of this similarity needs further biochemical studies.

Identification of synthetic lethal agents with CHD1 deficiency is expected to benefit those prostate cancer cases that harbor this aberration. In early clinical studies, patients with CHD1 deficient prostate cancer responded to PARP inhibitor and platinum-based therapy²⁴. However, the subclonal nature of CHD1 loss highlighted here may have considerable clinical consequences. Tumors with CHD1 loss in a significant subset of the cells may show significant response to HR deficiency directed therapy. HR deficiency associated mutational signatures are used to prioritize ovarian cancer patients for PARP inhibitor therapy, and a similar strategy may be considered for prostate cancer as well. However, as shown here, the HR deficiency associated mutational signatures are “diluted out” proportionally to the subclonality of CHD1 loss. Therefore, diagnostic cut-off values may need to be readjusted for CHD1 deficient cases. If only a smaller subset of tumor cells harbor CHD1 deficiency, then a synthetic lethal agent may have only a modest benefit in terms of tumor shrinkage. However, as it was suggested recently, CHD1 loss may play a key role developing enzalutamide resistance¹⁴. Therefore, it is possible that eliminating the subset of CHD1 deleted tumor cells, even if a minority, will significantly delay antiandrogen therapy resistance. Moreover, the majority of specimens analyzed for CHD1 deletions in this study represent treatment naïve primary prostate tumor specimens. It would be reasonable to expect clonal expansion of CHD1 deleted tumor cells in more advanced heavily treated metastatic and castration resistant prostate cancers (CRPC), which calls for further analysis. Therefore, agents with higher specificity for CHD1 deficiency, such as talazoparib or bleomycin, may be used as agents to stave off resistance to antiandrogen therapy. Considering the higher frequency of CHD1 loss in AA PC, such CHD1 directed therapy may stop the early development of more malignant clones and may reduce the racial differences in the overall outcome of prostate cancer.

All references and publications cited here are incorporated by reference in their entirely, including the references to gene sequence databases, such as Genbank, and the specific nucleotide sequences associated with the sequence identifier.

In this disclosure, “a” and “an” and similar phrases are to be interpreted as “at least one” or “one or more.” Similarly, any term that ends with the suffix “(s)” is to be interpreted as “at least one” or “one or more.” In this disclosure, the term “may” is to be interpreted as “may, for example.” In other words, the term “may” is indicative that the phrase following the term “may” is an example of one of a multitude of suitable possibilities that may, or may not, be employed to one or more of the various embodiments. The terms “including” and “comprising” should be interpreted as meaning “including, but not limited to”.

In this disclosure, a “therapeutically effective amount” or “effective amount” of a drug or pharmaceutical agent means an amount of the drug or pharmaceutical agent that results in a decrease in severity of disease symptoms, an increase in frequency and/or duration of disease symptom-free periods, prevention or reduction of likelihood of impairment or disability due to the disease affliction, or inhibition or delaying of the progression of disease.

For the sake of brevity and legibility, the present disclosure does not explicitly recite each and every permutation that may be obtained by choosing from the set of optional features. However, the present disclosure is to be interpreted as explicitly disclosing all such permutations. For example, a system described as having three optional features may be embodied in seven different ways, namely with just one of the three possible features, with any two of the three possible features or with all three of the three possible features.

While various embodiments have been described above, it should be understood that they have been presented by way of example, and not limitation. It will be apparent to persons skilled in the relevant art(s) that various changes in form and detail can be made therein without departing from the scope. In fact, after reading the above description, it will be apparent to one skilled in the relevant art(s) how to implement alternative embodiments. Thus, the present embodiments should not be limited by any of the above described exemplary embodiments.

Further, the purpose of the Abstract of the Disclosure is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract of the Disclosure is not intended to be limiting as to the scope in any way.

### REFERENCES

1. Siegel, R. L., Miller, K. D. & Jemal, A. Cancer Statistics, 2017. CA Cancer J Clin 67, 7-30 (2017).
2. Gaines, A. R. et al. The association between race and prostate cancer risk on initial biopsy in an equal access, multiethnic cohort. Cancer Causes Control 25, 1029-1035 (2014).
3. Chu, D. I. et al. Effect of race and socioeconomic status on surgical margins and biochemical outcomes in an equal-access health care setting: results from the Shared Equal Access Regional Cancer Hospital (SEARCH) database. Cancer 118, 4999-5007 (2012).
4. Khani, F. et al. Evidence for molecular differences in prostate cancer between African American and Caucasian men. Clin. Cancer Res. 20, 4925-4934 (2014).
5. Rosen, P. et al. Differences in frequency of ERG oncoprotein expression between index tumors of Caucasian and African American patients with prostate cancer. Urology 80, 749-753 (2012).
6. Sedarsky, J., Degon, M., Srivastava, S. & Dobi, A. Ethnicity and ERG frequency in prostate cancer. Nat Rev Urol 15, 125-131 (2018).
7. Petrovics, G. et al. A novel genomic alteration of LSAMP associates with aggressive prostate cancer in African American men. EBioMedicine 2, 1957-1964 (2015).
8. Koga, Y. et al. Genomic Profiling of Prostate Cancers from Men with African and European Ancestry. Clin Cancer Res 26, 4651-4660 (2020).
9. Mahal, B. A. et al. Racial Differences in Genomic Profiling of Prostate Cancer. N Engl J Med 383, 1083-1085 (2020).
10. Huang, F. W. et al. Exome Sequencing of African-American Prostate Cancer Reveals Loss-of-Function ERF Mutations. Cancer Discov 7, 973-983 (2017).
11. Faisal, F. A. et al. SPINK1 expression is enriched in African American prostate cancer but is not associated with altered immune infiltration or oncologic outcomes post-prostatectomy. Prostate Cancer Prostatic Dis 22, 552-559 (2019).
12. Burkhardt, L. et al. CHD1 is a 5q21 tumor suppressor required for ERG rearrangement in prostate cancer. Cancer Res. 73, 2795-2805 (2013).
13. Augello, M. A. et al. CHD1 Loss Alters AR Binding at Lineage-Specific Enhancers and Modulates Distinct Transcriptional Programs to Drive Prostate Tumorigenesis. Cancer Cell 35, 817-819 (2019).
14. Zhang, Z. et al. Loss of CHD1 Promotes Heterogeneous Mechanisms of Resistance to AR-Targeted Therapy via Chromatin Dysregulation. Cancer Cell 37, 584-598.e11 (2020).
15. Zhou, J. et al. Human CHD1 is required for early DNA-damage signaling and is uniquely regulated by its N terminus. Nucleic Acids Res. 46, 3891-3905 (2018).
16. Kari, V. et al. Loss of CHD1 causes DNA repair defects and enhances prostate cancer therapeutic responsiveness. EMBO Rep. 17, 1609-1623 (2016).
17. Hjorth-Jensen, K. et al. SPOP promotes transcriptional expression of DNA repair and replication factors to prevent replication stress and genomic instability. Nucleic Acids Res. 46, 9484-9495 (2018).
18. Baca, S. C. et al. Punctuated evolution of prostate cancer genomes. Cell 153, 666-677 (2013).
19. Ha, G. et al. TITAN: inference of copy number architectures in clonal cell populations from tumor whole-genome sequence data. Genome Res. 24, 1881-1893 (2014).
20. Han, M. et al. Biochemical (prostate specific antigen) recurrence probability following radical prostatectomy for clinically localized prostate cancer. J Urol. 169, 517-523 (2003).
21. Yuan, J. et al. Integrative comparison of the genomic and transcriptomic landscape between prostate cancer patients of predominantly African or European genetic ancestry. PLoS Genet. 16, e1008641 (2020).
22. Oesper, L., Mahmoody, A. & Raphael, B. J. THetA: inferring intra-tumor heterogeneity from high-throughput DNA sequencing data. Genome Biol. 14, R80 (2013).
23. Cun, Y., Yang, T.-P., Achter, V., Lang, U. & Peifer, M. Copy-number analysis and inference of subclonal populations in cancer genomes using Sclust. Nat Protoc 13, 1488-1501 (2018).
24. Shenoy, T. R. et al. CHD1 loss sensitizes prostate cancer to DNA damaging therapy by promoting error-prone double-strand break repair. Ann. Oncol. 28, 1495-1507 (2017).
25. Alexandrov, L. B. et al. Signatures of mutational processes in human cancer. Nature 500, 415-421 (2013).
26. Alexandrov, L. B. et al. The repertoire of mutational signatures in human cancer. Nature 578, 94-101 (2020).
27. Nik-Zainal, S. et al. Landscape of somatic mutations in 560 breast cancer whole-genome sequences. Nature 534, 47-54 (2016).
28. Póti, Á. et al. Correlation of homologous recombination deficiency induced mutational signatures with sensitivity to PARP inhibitors and cytotoxic agents. Genome Biol. 20, 240 (2019).
29. Telli, M. L. et al. Homologous Recombination Deficiency (HRD) Score Predicts Response to Platinum-Containing Neoadjuvant Chemotherapy in Patients with Triple-Negative Breast Cancer. Clin. Cancer Res. 22, 3764-3773 (2016).
30. Davies, H. et al. HRDetect is a predictor of BRCA1 and BRCA2 deficiency based on mutational signatures. Nat. Med. 23, 517-525 (2017).
31. Sztupinszki, Z. et al. Detection of Molecular Signatures of Homologous Recombination Deficiency in Prostate Cancer with or without BRCA1/2 Mutations. Clin. Cancer Res. 26, 2673-2680 (2020).
32. Zámborszky, J. et al. Loss of BRCA1 or BRCA2 markedly increases the rate of base substitution mutagenesis and has distinct effects on genomic deletions. Oncogene 36, 746-755 (2017).
33. Feng, W. et al. Genetic determinants of cellular addiction to DNA polymerase theta. Nat Commun 10, 4286 (2019).
34. Murai, J. & Pommier, Y. PARP Trapping Beyond Homologous Recombination and Platinum Sensitivity in Cancers. Annu. Rev. Cancer Biol. 3, 131-150 (2019).
35. Barbieri, C. E. et al. The mutational landscape of prostate cancer. Eur. Urol. 64, 567-576 (2013).
36. Yates, L. R. et al. Subclonal diversification of primary breast cancer revealed by multiregion sequencing. Nat. Med. 21, 751-759 (2015).
37. Linch, M. et al. Intratumoural evolutionary landscape of high-risk prostate cancer: the PROGENY study of genomic and immune parameters. Ann. Oncol. 28, 2472-2480 (2017).
38. Merseburger, A. S. et al. Limitations of tissue microarrays in the evaluation of focal alterations of bcl-2 and p53 in whole mount derived prostate tissues. Oncol Rep 10, 223-228 (2003).
39. Furusato, B. et al. ERG oncoprotein expression in prostate cancer: clonal progression of ERG-positive tumor cells and potential for ERG-based stratification. Prostate Cancer Prostatic Dis 13, 228-237 (2010).
40. Karczewski, K. J. et al. The ExAC browser: displaying reference data information from over 60 000 exomes. Nucleic Acids Res 45, D840-D845 (2017).
41. Quinlan, A. R. & Hall, I. M. BEDTools: a flexible suite of utilities for comparing genomic features. Bioinformatics 26, 841-842 (2010).
42. Li, H. et al. The Sequence Alignment/Map format and SAMtools. Bioinformatics 25, 2078-2079 (2009).
43. Favero, F. et al. Sequenza: allele-specific copy number and mutation profiles from tumor sequencing data. Ann. Oncol. 26, 64-70 (2015).
44. McKenna, A. et al. The Genome Analysis Toolkit: a MapReduce framework for analyzing next-generation DNA sequencing data. Genome Res 20, 1297-1303 (2010).
45. Li, H. & Durbin, R. Fast and accurate short read alignment with Burrows-Wheeler transform. Bioinformatics 25, 1754-1760 (2009).
46. Li, Q. & Wang, K. InterVar: Clinical Interpretation of Genetic Variants by the 2015 ACMG-AMP Guidelines. Am. J. Hum. Genet. 100, 267-280 (2017).

1. A nucleic acid probe for hybridization to chromosomal DNA, comprising at least two different nucleic acid molecules from the same chromosomal region of CHD1, where said nucleic acid molecules are present in different proportions from each other.

2. The nucleic acid probe of claim 1, comprising at least 10 nucleic acid molecules, where at least three of said 10 molecules are present in different proportions from each other and in different proportions that they occur in nature.

3. The nucleic acid probe of claim 1, where the probe is for detecting a deletion in the CHD1 gene and the probe comprises molecules having sequences from the CHD1 gene, where the molecules are present in different proportions from each other and in different proportions than those in which they occur in nature.

4. The nucleic acid probe of claim 1, where the probe is for detecting a deletion in the CHD1 gene and the probe comprises molecules having sequences from the CHD1 gene, where the molecules are present in different proportions from each other and in different proportions that they occur in nature.

5. The nucleic acid probe of claim 1, further comprising molecules having sequences from the LSAMP gene deleted in patients with prostate cancer, where the molecules are present in different proportions from each other and in different proportions that they occur in nature.

6. The nucleic acid probe of claim 1, further comprising molecules having sequences from the PTEN gene deleted in patients with prostate cancer, where the molecules are present in different proportions from each other and in different proportions that they occur in nature.

7. The nucleic acid probe of claim 1, further comprising at least two molecules having sequences from FIG. 3.

8. The nucleic acid probe of claim 1, further comprising at least two molecules having sequences from Table 1.

9. The nucleic acid probe of claim 1, further comprising five molecules having sequences from Table 1.

10. A method of treating prostate cancer in a subject, comprising:
assessing whether the subject's prostate cancer is associated with CHD1 deficiency by performing a FISH assay using a nucleic acid probe of claim 1 on a nucleic acids sample obtained from the subject; and
if it is determined that the subject's prostate cancer is associated with CHD1 deficiency, administering an effective amount of at least one of Talazoparib and Olaparib to the subject.

11. A method of treating prostate cancer in a subject, comprising:
assessing whether the subject's prostate cancer is associated with CHD1 deficiency based on a nucleic acids sample obtained from the subject; and
if it is determined that the subject's prostate cancer is associated with CHD1 deficiency, administering an effective amount of bleomycin to the subject.

12. The method of claim 11, further comprising administering to the subject a PARP inhibitor.

13. The method of claim 12, wherein the PARP inhibitor is selected from the group consisting of Olaparib and Talazoparib.

14. The method of claim 11, wherein assessing whether the subject's prostate cancer is associated with CHD1 deficiency comprises performing a FISH assay using a nucleic acid probe of claim 1 on the nucleic acids sample obtained from the subject.

15. The method of claim 1, wherein the nucleic acids sample is obtained from a cancerous prostate tissue of the subject.

16. The method of claim 1, wherein the nucleic acids sample is obtained from circulating tumor cells or cell-free circulating nucleic acids from the subject.
